SEQUENCE LISTING (2 sequences)

SEQ ID NO: 1 - protein, 18 aa, Artificial
  Attenuating mutation insertion sequence
ITSMDSTSSGPSSLEIVD

SEQ ID NO: 2 - DNA, 20 bp, Artificial
  Vaccination oligonucleotide
tccatgacgttcctgacgtt

(19) United States
(12) Patent Application Publication
(10) Pub. No.: US 2011/0070260 A1
(43) Pub. Date: Mar. 24, 2011

(54) Multivalent Immunogenic Compositions Against Noroviruses and Methods of Use

(76) Inventors: Ralph S. Baric, Haw River, NC (US); Anna LoBue, South San Francisco, CA (US); Robert Edward Johnston, Chapel Hill, NC (US); Joseph Thompson, Gainesville, FL (US); Lisa Lindesmith, Apex, NC (US)

(21) Appl. No.: 12/875,369

(22) Filed: Sep. 3, 2010

Related U.S. Application Data
(60) Provisional application No. 61/240,871, filed on Sep. 9, 2009.

(51) Int. Cl. A61K 39/125; A61P 37/04; A61P 31/14

(52) U.S. Cl. 424/202.1; 424/216.1

(57) ABSTRACT

The invention provides immunogenic formulations comprising virus-like particles (VLPs) from two or more genoclusters and/or strains of norovirus in a pharmaceutically acceptable carrier. In representative embodiments, the formulation also comprises an adjuvant, for example, a viral adjuvant or CpG. The invention also provides methods of inducing an immune response to one or more noroviruses.

### RELATED APPLICATION INFORMATION

This application claims the benefit of U.S. Provisional Patent Application No. 61/240,871, filed Sep. 9, 2009, the entire disclosure of which is incorporated by reference herein.

### STATEMENT OF FEDERAL SUPPORT

This invention was supported in part by funding provided under Grant No. RO1AI056351 from the National Institute of Allergy and Infectious Diseases. The United States government has certain rights in this invention.

### FIELD OF THE INVENTION

The present invention relates to immunogenic compositions and methods for inducing an immune response against noroviruses.

### BACKGROUND OF THE INVENTION

The development of an effective Norovirus vaccine will be facilitated by the ability to protect against infection with multiple norovirus genoclusters and/or strains, especially those contemporary strains that are currently causing outbreaks of disease. The Norovirus genome is an ˜7.8 Kb single-stranded, plus-sensed RNA encoding three open reading frames (ORF): a 1738 amino acid polymerase region (ORF1), a major capsid protein of ˜530 amino acids (ORF2), and a 212 amino acid minor capsid protein (ORF3). Noroviruses are subdivided into five genogroups (GI to GV) that differ by >50% with respect to the primary amino acid sequence in ORF2. The GI and GII genogroups cause about 5% and 95%, respectively, of the recent norovirus outbreaks. GI and GII are further subdivided into ˜8-9 and ˜19 genoclusters, respectively, that differ by at least about 20% amino acid variability in ORF2. However, norovirus serogroups are not well defined as infection elicits only low level cross-reactive antibody responses across genogroups.

Noroviruses are the most important cause of food-borne gastroenteritis worldwide and cause about 85 to 96% of selected outbreaks of acute non-bacterial gastroenteritis in the United States. Outbreaks occur in communities, families, recreational facilities, retirement communities, day care centers, schools, cruise and military ships, and military hospitals. These viruses are also the most frequent cause of acute gastroenteritis following ingestion of raw shellfish, are the second most important cause of severe viral gastroenteritis in young children, and may cause about 20% of endemic gastroenteritis in families. Heralded as the “stomach flu,” retirement community outbreaks are pervasive and can result in 1-2% mortality rates in the elderly. During outbreaks, the sheer numbers of incontinent patients can paralyze staff and compromise institutional operations. Moreover, noroviruses cause some 230 million cases of diarrhea each year in the United States, resulting in some 50,000 hospitalizations each year. Outbreaks of norovirus gastroenteritis are common on military ships, compromising routine ship operations. Deployed military personnel are at high risk because crowded conditions and poor sanitation facilitates rapid person-to-person transmission in combat situations. Outbreaks on cruise ships are common, including repeat infections with the same strain. The 85 billion dollar cruise industry served about 12 million people in 2006, a number expected to exceed 27 million by 2020. Today, the industry absorbs significant losses from disinfection costs, cancellations, travel delays, patient costs, legal fees, bad press and discount vouchers. Norovirus infections also cause about 30 to 50% of travelers' diarrhea, persist for months in immunosuppressed people, are Category B biodefense pathogens, and are on the United States Environmental Protection Agency's “candidate contaminant list” for the regulation of drinking waters. Worldwide, about 200,000 children die each year from norovirus induced gastroenteritis.

Noroviruses are transmitted via ingestion of fecally contaminated food and water, exposure to contaminated fomites, aerosolized vomitus, and direct person-to-person contact. There are no currently approved vaccines or therapeutics.

Accordingly, there is a need in the art for improved immunogenic compositions and methods to induce immune responses and provide protection against noroviruses.

### SUMMARY OF THE INVENTION

As a first aspect, the invention provides an immunogenic formulation comprising virus-like particles (VLPs) from two or more genoclusters and/or strains of norovirus in a pharmaceutically acceptable carrier. Optionally, the immunogenic formulation further comprises an adjuvant. The adjuvant can be a viral adjuvant, for example, an alphavirus adjuvant.

As a further aspect, the invention provides a method of producing an immune response against two or more noroviruses in a subject, the method comprising administering an immunogenically effective amount of a formulation of the invention to the subject.

As still a further aspect, the invention provides a method of protecting a subject from infection by two or more noroviruses, the method comprising administering a formulation of the invention to the subject in an amount effective to protect the subject from infection by the two or more noroviruses.

Still further, the invention provides a method of producing an immune response against two or more noroviruses in a subject, the method comprising administering to the subject:

(a) an immunogenically effective amount of a formulation of the invention; and

(b) an alphavirus adjuvant comprising: a modified alphavirus genomic nucleic acid that lacks sequences encoding the alphavirus structural proteins required for production of new alphavirus particles; wherein the modified alphavirus genome does not comprise a heterologous nucleic acid sequence encoding the VLPs from two or more genoclusters and/or strains of norovirus.

The invention also encompasses a method of protecting a subject from infection by two or more noroviruses, the method comprising administering to the subject:

(a) a formulation of the invention in an amount effective to protect the subject from infection by the two or more noroviruses; and

(b) an alphavirus adjuvant comprising: a modified alphavirus genomic nucleic acid that lacks sequences encoding the alphavirus structural proteins required for production of new alphavirus particles; wherein the modified alphavirus genome does not comprise a heterologous nucleic acid sequence encoding the VLPs from two or more genoclusters and/or strains of norovirus.

These and other aspects of the invention are set forth in more detail below.

### BRIEF DESCRIPTION OF THE DRAWINGS

FIG. 1. Effective VLP dose for null VRP adjuvant activity. Mice were immunized with 10⁵ IU of null VRPs and NV VLPs at doses of 0.02 μg, 0.2 μg, 2 μg, and 10 μg. Fecal extracts were prepared, and anti-NV IgG and IgA were quantitated by ELISA (A). Sera were also tested for anti-NV IgG and interference of H type 3 binding to NV VLPs (B and C). One asterisk (*) is representative of P values of <0.05, and three asterisks (***) are representative of P values of <0.001.

FIG. 2. Homotypic antibody responses following monovalent vaccination with and without adjuvant. Sera from mice immunized with NV or LV VLPs alone or in conjunction with CpG or null VRP adjuvants were analyzed for anti-NV or anti-LV IgG, respectively, by ELISA (A). Serially diluted antisera were also tested for blockade of H type 3 binding to NV VLPs (B) and LV VLPs (C). Two asterisks (**) are representative of P values of <0.01, and three asterisks (***) are representative of P values of <0.001.

FIG. 3. Antibody responses following multivalent vaccination with or without adjuvant. Sera from animals immunized with multivalent VLP vaccines either alone or in conjunction with CpG or null VRP adjuvant were analyzed for IgG reactivity to NV or LV VLPs by ELISA (A). GI+/GII+ groups received NV and LV VLPs as a vaccine component; GI−/GII− groups did not. Serially diluted sera were also tested for interference of H type 3 binding to NV VLPs (B) and LV VLPs (C). One asterisk (*) is representative of P values of <0.05, and three asterisks (***) are representative of P values of <0.001.

FIG. 4. Antibody responses to monovalent, genogroup-specific, and cumulative VLP cocktail vaccines coadministered with null VRP adjuvant. Sera from animals immunized with null VRP and monovalent, genogroup-specific multivalent, cumulative multivalent, or heterotypic monovalent VLP vaccines with or without NV or LV VLPs as a vaccine component were analyzed for IgG reactivity to NV or LV VLPs by ELISA (A). Serially diluted sera were also tested for interference of H type 3 binding to NV VLPs (B) and LV VLPs (C). One asterisk (*) is representative of P values of <0.05, two asterisks (**) are representative of P values of <0.01, and three asterisks (***) are representative of P values of <0.001.

FIG. 5. Serum IgG cross-reactivity profile. Antisera from mice immunized with each monovalent or multivalent VLP vaccine coadministered with null VRP adjuvant were analyzed for IgG cross-reactivity to the VLP panel by ELISA.

FIG. 6. IgG subtypes in serum following monovalent, multivalent, and adjuvanted vaccination. Mice immunized with monovalent (A and B) and multivalent vaccines with (C and D) or without (E and F) NV (left panels) or LV (right panels) VLPs and with or without adjuvant were analyzed for IgG1 and IgG2a serum antibody subtype responses by ELISA. Subtype responses to increasing amounts of VLPs are shown in panels G and H.

FIG. 7. Viral titers and antibody responses following MNV challenge in monovalent, multivalent, and adjuvant-vaccinated mice. Mice immunized with adjuvanted or unadjuvanted monovalent MNV VLP or multivalent VLPs±MNV VLPs were challenged with MNV, and tissues were harvested 3 days postinfection. Plaque assays were performed on homogenized spleen, MLNs, and distal ileum to determine viral titers (A). Vaccination and challenge in all null VRP recipient groups were repeated, and MNV titers were determined in corresponding tissues (B). Serum IgG reactivity to MNV VLPs was determined by ELISA (C). One asterisk (*) is representative of P values of <0.05, two asterisks (**) are representative of P values of <0.01, and three asterisks (***) are representative of P values of <0.001.

FIG. 8. MNV infection of naïve mice following transfer of immune T-cell subsets or sera. Wild-type mice were immunized three times with MNV VLPs coadministered with null VRPs. Unimmunized controls were treated in parallel. Two weeks after the final boost, sera and CD4⁺ and CD8⁺ splenocytes were harvested and purified. Sera, CD4⁺ splenocytes, or CD8⁺ splenocytes were passively or adoptively transferred to naïve SCID knockout mice or wild-type mice. Twenty-four hours posttransfer, mice were infected with MNV-1, and tissues were harvested 3 days postinfection. Spleens were evaluated for MNV titers by plaque assay (A). MNV-specific serum IgG in serum donor and recipient mice was measured by ELISA (B). Nonimmune mice had no detectable MNV antibody and were assigned values that were half the lower limit of detection per assay. ***, P<0.001.

### DETAILED DESCRIPTION OF THE INVENTION

The present invention will be described with reference to the accompanying drawings, in which representative embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference in their entirety.

### I. Definitions.

The following terms are used in the description herein and the appended claims:

The singular forms “a,” “an” and “the” are intended to include the plural forms as well, unless the context clearly indicates otherwise.

Furthermore, the term “about,” as used herein when referring to a measurable value such as an amount or the length of a polynucleotide or polypeptide sequence, dose, time, temperature, and the like, is meant to encompass variations of ±20%, ±10%, ±5%, ±1%, ±0.5%, or even ±0.1% of the specified amount.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate, if the specification states that a virus like particle comprises components A, B and C, it is specifically intended that in representative embodiments any of A, B or C, or a combination thereof, can be omitted and disclaimed.

As used herein, the transitional phrase “consisting essentially of” is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP §2111.03. Thus, the term “consisting essentially of” as used herein should not be interpreted as equivalent to “comprising.”

As used herein, the term “concurrent” or “concurrently” means sufficiently close in time to produce a combined effect (that is, simultaneously or two or more events occurring within a short time period before or after each other).

As used herein, “nucleic acid” encompasses both RNA and DNA, including cDNA, genomic DNA, synthetic (e.g., chemically synthesized) DNA and chimeras of RNA and DNA. The nucleic acid may be double-stranded or single-stranded. Where single-stranded, the nucleic acid may be a sense strand or an antisense strand. The nucleic acid may be synthesized using nucleotide analogs or derivatives (e.g., inosine or phosphorothioate nucleotides). Such nucleotides analogs or derivatives can be used, for example, to prepare nucleic acids that have altered base-pairing abilities or increased resistance to nucleases.

As used herein, an “isolated” polynucleotide and similar terms (e.g., an “isolated nucleic acid,” “isolated DNA” or an “isolated RNA”) mean a polynucleotide at least partially separated from at least some of the other components of the naturally occurring organism or virus, for example, the cell or viral structural components or other polypeptides commonly found associated with the polynucleotide.

The term “heterologous nucleic acid” is a well-known term of art and would be readily understood by one of skill in the art to be a nucleic acid that is not normally present within the host cell, virus or vector into which it has been introduced. A heterologous nucleic acid can also be an additional copy of a nucleic acid that is endogenous to the cell, virus or vector, where the additional copy is introduced into the cell, virus or vector. In embodiments of the invention, the heterologous nucleic acid can encode a polypeptide or functional untranslated RNA of interest.

The heterologous nucleic acid can be associated with appropriate expression control sequences, e.g., transcription/translation control signals and polyadenylation signals.

It will be appreciated that a variety of promoter/enhancer elements can be used depending on the level and tissue-specific expression desired. The promoter can be constitutive or inducible (e.g., the metallothionein promoter or a hormone inducible promoter), depending on the pattern of expression desired. The promoter can be native or foreign and can be a natural or a synthetic sequence. By foreign, it is intended that the promoter is not found in the virus into which the promoter is introduced. The promoter is generally chosen so that it will function in the target cell(s) of interest. In particular embodiments, the heterologous nucleotide sequence is operably associated with a promoter that provides high level expression of the heterologous nucleotide sequence. In some embodiments, the promoter is an alphavirus subgenomic 26S promoter (preferably, a VEE, Sindbis, Girdwood or TR339 26S subgenomic promoter), which can have a wild type or modified sequence (e.g., can be a modified 26S promoter sequence having reduced activity [e.g., transcriptional activity] as described herein).

Inducible expression control elements can be used in those applications in which it is desirable to provide regulation over expression of the heterologous nucleic acid sequence. Inducible promoters/enhancer elements include tissue-specific or—preferred promoter/enhancer elements, which further includes, but is not limited to, muscle specific or preferred (including cardiac, skeletal and/or smooth muscle), neural tissue specific or preferred (including brain-specific or preferred), eye specific or preferred (including retina-specific or preferred and cornea-specific or preferred), liver specific or preferred, bone marrow specific or preferred, pancreatic specific or preferred, spleen specific or preferred, and lung specific or preferred promoter/enhancer elements. Other inducible promoter/enhancer elements include hormone-inducible and metal-inducible elements, examples of which include but are not limited to a Tet on/off element, a RU486-inducible promoter, an ecdysone-inducible promoter, a rapamycin-inducible promoter, and a metallothionein promoter.

Moreover, specific initiation signals are generally used for efficient translation of inserted polypeptide coding sequences. These translational control sequences, which can include the ATG initiation codon and adjacent sequences, can be of a variety of origins, both natural and synthetic. In embodiments of the invention wherein there are two or more heterologous nucleic acids to be transcribed, the transcriptional units can be operatively associated with separate promoters or with a single upstream promoter and one or more downstream internal ribosome entry site (IRES) sequences (e.g., the picornavirus EMC IRES sequence).

As used herein, the terms “express,” “expresses,” “expressed” or “expression,” and the like, with respect to a nucleic acid sequence (e.g., RNA or DNA) indicates that the nucleic acid sequence is transcribed and, optionally, translated. Thus, a nucleic acid sequence may express a polypeptide of interest or a functional untranslated RNA.

A “functional untranslated RNA” includes, for example, interfering RNA (e.g., siRNA) or antisense RNA.

Subjects according to the present invention include both avians and mammals, including male and/or female subjects. Mammalian subjects include but are not limited to humans, non-human mammals, non-human primates (e.g., monkeys, chimpanzees, baboons, etc.), dogs, cats, mice, hamsters, rats, horses, cows, pigs, rabbits, sheep and goats. Avian subjects include but are not limited to chickens, turkeys, ducks, geese, quail, and pheasant, and birds kept as pets (e.g., parakeets, parrots, macaws, cockatoos, and the like). In particular embodiments, the subject is a laboratory animal. Subjects include infants, juveniles, adolescents, adults and/or geriatric subjects.

By the terms “treat,” “treating” or “treatment of” (and grammatical variations thereof) it is meant that the severity of the subject's condition is reduced, at least partially improved or stabilized and/or that some alleviation, mitigation, decrease or stabilization in at least one clinical symptom or parameter is achieved and/or there is a delay in the progression of the disease or disorder. In embodiments, the invention may be practiced to treat existing norovirus infection, e.g., in geriatric and/or immunosuppressed populations.

The terms “prevent,” “preventing” and “prevention” (and grammatical variations thereof) refer to avoidance, reduction and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present invention.

An “effective amount,” as used herein, refers to an amount that imparts a desired effect, which is optionally a therapeutic or prophylactic effect.

A “treatment effective” amount as used herein is an amount that is sufficient to treat (as defined herein) the subject. Those skilled in the art will appreciate that the therapeutic effects need not be complete or curative, as long as some benefit is provided to the subject.

A “prevention effective” amount as used herein is an amount that is sufficient to prevent (as defined herein) the disease, disorder and/or clinical symptom in the subject. Those skilled in the art will appreciate that the level of prevention need not be complete, as long as some benefit is provided to the subject.

The terms “vaccination” or “immunization” are well-understood in the art, and are used interchangeably herein unless otherwise indicated. For example, the terms vaccination or immunization can be understood to be a process that increases an organism's immune response to antigen and therefore to resist, reduce or overcome infection. In the case of the present invention, vaccination or immunization against noroviruses increases the organism's immune response to resist, reduce or overcome infection by two or more noroviruses.

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in IMMUNOLOGY: BASIC PROCESSES 117 (Joseph A. Bellanti ed., 1985). Alternatively stated, an active immune response is mounted by the host after exposure to immunogens by infection or by vaccination. Active immunity can be contrasted with passive immunity, which is acquired through the “transfer of preformed substances (antibody, transfer factor, thymic graft, interleukin-2) from an actively immunized host to a non-immune host.” Id.

The terms “protective” immune response or “protective” immunity (and similar terms) as used herein indicates that the immune response confers some benefit to the subject in that it prevents or reduces the incidence and/or severity and/or duration of disease.

By “mucosal immune response” it is meant an immune response (cellular and/or humoral) that is detectable and resident at a mucosal surface(s) of the host (e.g., the respiratory tract, the reproductive tract, the urinary tract, the gastrointestinal tract). Typically, but not necessarily, a mucosal immune response is accompanied by production of antigen-specific IgA and/or IgG molecules.

By “systemic immune response” it is meant an immune response (cellular, mucosal and/or humoral) that is detectable in blood, mucosal sites (e.g., gut secretions and stool) and/or lymphoid tissue (e.g., spleen and lymph nodes).

As used herein, the term “adjuvant” has its ordinary meaning as understood by those in the art. For example, an adjuvant can be defined as a substance that increases the ability of an immunogen (i.e., antigen) to stimulate an immune response against the immunogen in the subject. In particular embodiments, the adjuvant increases the immune response against the immunogen by at least about 2, 3, 4, 5, 10, 15, 20, 30, 40, 50, 60, 75, 100, 150, 500, 1000-fold or more. In other embodiments, the adjuvant reduces the amount of immunogen required to achieve a particular level of immune response (cellular and/or humoral and/or mucosal), e.g., a reduction of at least about 15%, 25%, 35%, 50%, 65%, 75%, 80%, 85%, 90%, 95%, 98% or more. An adjuvant can further be a substance that prolongs the time over which an immune response, optionally protective immune response, is sustained (e.g., by at least about a 2-fold, 3-fold, 5-fold, 10-fold, 20-fold longer time period or more).

An “adjuvant effective amount” is an amount of the adjuvant that is sufficient to enhance or stimulate the active immune response (cellular and/or humoral) mounted by the host. In particular embodiments, the active immune response (e.g., humoral and/or cellular immune response) by the host is enhanced by at least about 2, 3, 4, 5, 10, 15, 20, 30, 40, 50, 60, 75, 100, 150, 500, 1000-fold or more. In other embodiments, an “adjuvant effective amount” is an amount of the adjuvant that reduces the amount of antigen required to achieve a specified level of immunity (cellular and/or humoral), for example, a reduction of at least about 15%, 25%, 35%, 50%, 65%, 75%, 80%, 85%, 90%, 95%, 98% or more in the amount of antigen. As a further option, an “adjuvant effective amount” can refer to an amount of the adjuvant that accelerates the induction of the immune response in the host and/or reduces the need for booster immunizations to achieve protection or neutralization. As yet another alternative, an “adjuvant effective amount” can be an amount that prolongs the time period over which an immune response, optionally a protective immune response, is sustained (e.g., by at least about a 2-fold, 3-fold, 5-fold, 10-fold, 20-fold longer time period or more).

As used herein, the term “virus like particle” (VLP) indicates an assembled structure formed by and comprising, consisting essentially of, or consisting of one or more virus structural proteins, but lacking the naturally occurring viral nucleic acid. In embodiments of the invention, the VLP does not comprise any nucleic acid. Accordingly, VLPs are nonreplicating and noninfectious. The VLP will generally retain one or more of the antigenic determinants of the intact virus so that it can effectively induce an immune response that will provide protection against the infectious norovirus. In embodiments of the invention, a norovirus VLP comprises, consists essentially of, or consists of the norovirus major (ORF2) and/or minor (ORF3) capsid proteins. The capsid gene is divided into three discrete regions called the C-shell domain (inner core) and the P domain (protruding domain), which is further subdivided into the P1 (stalk) and P2 (surface exposed protrusion) subdomains. In embodiments of the invention, the norovirus VLP comprises, consists essentially of, or consists of the P1 and P2 domains (i.e., the C-shell domain is removed from the capsid gene). In other embodiments of the invention, the norovirus VLP comprises, consists essentially of, or consists of the P2 domain (i.e., the C-shell domain and P1 subdomain are removed from the capsid gene). Those skilled in the art will appreciate that the structural proteins can comprise modifications, for example, to enhance immunogenicity and/or stability and/or to facilitate detection and/or purification. In other embodiments, the polypeptide components of the VLP (e.g. the P domain or portions therein), can be linked to other carriers (e.g., protein, haptens, adjuvants, etc.) to stabilize the VLP or subparticle presentation and/or improve immunogenicity. In other instances, portions of Norovirus capsid proteins can be blended together to form chimeric VLPs harboring resident epitopes from two or more different genoclusters.

Methods of producing norovirus VLPs are known in the art. For example, VLPs can be expressed from virus vectors (e.g., alphaviruses, baculoviruses) or in yeast or microbial expression systems.

The term “norovirus” has its conventional meaning in the art and includes any virus now known or later identified as a norovirus by the International Committee on Taxonomy of Viruses (ICTV). Noroviruses are divided into at least five genogroups (GI, GII, GIII, GIV and GV), which are in turn classified into 30 genoclusters or more as the discovery of new genoclusters is occurring each year and new genoclusters can emerge by mutation and/or recombination driven processes and/or emergence from animal reservoirs. Genogroups GI, GII and GIV are known to infect humans, although GI and GII account for most incidents of human disease.

Examples of GI to GV norovirus genoclusters include without limitation:
GI Genogroup: GI.1 (Norwalk virus), GI.2 (Southampton virus), GI.3 (Desert Shield virus), GI.4 (Chiba virus), GI.5, GI.6, GI.7 and GI.8.
GII Genogroup: GII.1 (Hawaii virus), GII.2 (Snow Mountain virus), GII.3 (Toronto virus), GII.4 (Lordsdale virus), GII.6, GII.7, GII.8, CII.9, GII.10, GII.11, GII.13 (M7), GII.14, GII.17, GII.18 and GII.b.
GIII Genogroup: GIII.1 (bovine) and GIII.2 (bovine).
GIV Genogroup: GIV.1
GV Genogroup: GV.1 (murine)

Within each genocluster are individual strains (genotypes). Some genoclusters are relatively static, while others such as the GII.4 genocluster rapidly evolve (see, e.g., Lindesmith et al. Mechanisms of GII.4 norovirus persistence in human populations. PLoS Medicine. 5(2): e31 (2008)).

The term “aiphavirus” has its conventional meaning in the art, and includes Eastern Equine Encephalitis virus (EEE), Venezuelan Equine Encephalitis virus (VEE), Everglades virus, Mucambo virus, Pixuna virus, Western Encephalitis virus (WEE), Sindbis virus, South African Arbovirus No. 86 (S.A.AR86), Girdwood S. A. virus, Ockelbo virus, Semliki Forest virus, Middelburg virus, Chikungunya virus, O′Nyong-Nyong virus, Ross River virus, Barmah Forest virus, Getah virus, Sagiyama virus, Bebaru virus, Mayaro virus, Una virus, Aura virus, Whataroa virus, Babanki virus, Kyzlagach virus, Highlands J virus, Fort Morgan virus, Ndumu virus, Buggy Creek virus, and any other virus now known or later classified by the International Committee on Taxonomy of Viruses (ICTV) as an alphavirus.

Alphavirus particles comprise the alphavirus structural proteins assembled to form an enveloped nucleocapsid structure. As known in the art, alphavirus structural subunits consisting of a single viral protein, capsid, associate with themselves and with the RNA genome to form the icosahedral nucleocapsid, which is then surrounded by a lipid envelope covered with a regular array of transmembranal protein spikes, each of which consists of a heterodimeric complex of two glycoproteins, E1 and E2 (See Paredes et al., (1993) Proc. Natl. Acad. Sci. USA 90, 9095-99; Paredes et al., (1993) Virology 187, 324-32; Pedersen et al., (1974) J. Virol. 14:40). The wild-type alphavirus genome is a single-stranded, messenger-sense RNA, modified at the 5′-end with a methylated cap, and at the 3′-end with a variable-length poly (A) tract. The viral genome is divided into two regions: the first encodes the nonstructural or replicase proteins (nsP1-nsP4) and the second encodes the viral structural proteins (Strauss and Strauss, Microbiological Rev. (1994) 58:491-562).

As used herein, the term “polypeptide” encompasses both peptides and proteins.

A “polypeptide of interest” as used herein is a polypeptide that is desirably introduced and/or expressed in a subject, e.g., because of its biological and/or antigenic properties and includes reporter polypeptides, therapeutic polypeptides, enzymes, growth factors, immunomodulatory polypeptides, and immunogenic polypeptides.

An “isolated” polypeptide means a polypeptide that is separated or substantially free from at least some of the other components of the naturally occurring organism or virus, for example, the cell or viral structural components or other polypeptides or nucleic acids commonly found associated with the polypeptide.

The term “viral structural protein(s)” as used herein refers to one or more of the proteins that are constituents of a functional virus particle. The norovirus structural proteins are the major (ORF2) and minor (ORF3) capsid subunits, which assemble to form a non-enveloped virion. The alphavirus structural proteins include the capsid protein, E1 glycoprotein, E2 glycoprotein, E3 protein and 6K protein. The alphavirus particle comprises the alphavirus structural proteins assembled to form an enveloped nucleocapsid structure. As known in the art, alphavirus structural subunits consisting of a single viral protein, capsid, associate with themselves and with the RNA genome to form the icosahedral nucleocapsid, which is then surrounded by a lipid envelope covered with a regular array of transmembranal protein spikes, each of which consists of a heterodimeric complex of two glycoproteins, E1 and E2 (See Paredes et al., (1993) Proc. Natl. Acad. Sci. USA 90, 9095-99; Paredes et al., (1993) Virology 187, 324-32; Pedersen et al., (1974) J. Virol. 14:40).

Further, the term “viral structural protein” or similar terms include, without limitation, naturally occurring viral structural proteins and modified forms and active fragments thereof that induce an immune response in a subject, optionally a protective immune response, against one or more naturally occurring viral structural proteins. For example, a native structural protein can be modified to increase safety and/or immunogenicity and/or as a result of cloning procedures or other laboratory manipulations. Further, in embodiments of the invention, the amino acid sequence of the modified form of the viral structural protein can comprise one, two, three or fewer, four or fewer, five or fewer, six or fewer, seven or fewer, eight or fewer, nine or fewer, or ten or fewer modifications as compared with the amino acid sequence of the naturally occurring viral structural protein and induce an immune response (optionally a protective immune response) against a naturally occurring viral structural protein in the host. Suitable modifications encompass deletions (including truncations), insertions (including N- and/or C-terminal extensions) and amino acid substitutions, and any combination thereof. In representative embodiments, the viral structural protein is substantially similar at the amino acid level to the amino acid sequence of a naturally occurring viral structural protein and induces an immune response (optionally a protective immune response) against the virus in a host.

In embodiments of the invention, a “modified” viral structural protein induces an immune response in a host (e.g., IgG and/or IgA that react with the native viral structural protein), optionally a protective immune response, that is at least about 50%, 75%, 80%, 85%, 90%, or 95% or more of the immune response induced by the native viral structural protein, or induces an immune response that is the same as or essentially the same as the native viral structural protein, or induces an immune response that is even greater than the immune response induced by the native viral structural protein.

As used herein, an amino acid sequence that is “substantially identical” or “substantially similar” to a reference amino acid sequence is at least about 75%, 80%, 85%, 90%, 95%, 97%, 98% or 99% identical or similar, respectively, to the reference amino acid sequence.

Methods of determining sequence similarity or identity between two or more amino acid sequences are known in the art. Sequence similarity or identity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J. Mol. Biol. 48, 443 (1970), by the search for similarity method of Pearson & Lipman, Proc. Natl. Acad. Sci. USA 85, 2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12, 387-395 (1984), or by inspection.

Another suitable algorithm is the BLAST algorithm, described in Altschul et al., J. Mol. Biol. 215, 403-410, (1990) and Karlin et al., Proc. Natl. Acad. Sci. USA 90, 5873-5787 (1993). A particularly useful BLAST program is the WU-BLAST-2 program which was obtained from Altschul et al., Methods in Enzymology, 266, 460-480 (1996); http://blast.wustl/edu/blast/README.html. WU-BLAST-2 uses several search parameters, which are optionally set to the default values. The parameters are dynamic values and are established by the program itself depending upon the composition of the particular sequence and composition of the particular database against which the sequence of interest is being searched; however, the values may be adjusted to increase sensitivity.

Further, an additional useful algorithm is gapped BLAST as reported by Altschul et al., (1997) Nucleic Acids Res. 25, 3389-3402.

In embodiments of the invention, an “active fragment” of a viral structural protein is at least about 20, 30, 50, 75, 100, 125, 150, 200, 250, 300, 350, 400 or more contiguous amino acids and/or less than about 1000, 900, 800, 750, 700, 650, 600, 550, 500, 475, 450, 425, 400, 375, 350, 325, 300, 275, 250, 225, 200, 175, 150, 125, 100, 75 or 50 contiguous amino acids (including any combination of the foregoing as long as the lower limit is less than the upper limit) that can assemble into the VLP, and the VLP can induce an immune response (e.g., IgG and/or IgA that react with the viral structural protein), optionally a protective immune response, against the virus in a host. In particular embodiments, the VLP comprising the active fragment induces an immune response in a host, optionally a protective immune response, that is at least about 50%, 75%, 80%, 85%, 90%, or 95% or more of the immune response induced by a VLP comprising the full-length viral structural protein, or induces an immune response that is the same as or essentially the same as a VLP comprising the full-length viral structural protein.

A “viral genomic nucleic acid” and similar terms include wild-type viral genomes as well as recombinant and/or other modified forms (e.g., one or more attenuating mutations, deletions, insertions or otherwise modified viral genomes). The viral genomic nucleic acid can be a propagation-incompetent, but replication-competent, replicon as described herein. An “alphavirus genomic RNA” indicates the alphavirus RNA transcript, including recombinant and/or other modified forms. The wild-type alphavirus genome is a single-stranded, messenger-sense RNA, modified at the 5′-end with a methylated cap, and at the 3′-end with a variable-length poly (A) tract. The viral genome is divided into two regions: the first encodes the nonstructural or replicase proteins (nsP1-nsP4) and the second encodes the viral structural proteins (Strauss and Strauss, Microbiological Rev. (1994) 58:491-562). As used herein, the term “alphavirus genomic RNA” encompasses wild-type and recombinant alphavirus genomes (e.g., containing a heterologous nucleic acid sequence) as well as alphaviral genomes containing one or more attenuating mutations, deletions, insertions, and/or otherwise modified alphaviral genomes. For example, the “alphavirus genomic RNA” may be modified to form a double-promoter molecule or a replicon (each as described herein). The viral or alphavirus genomic nucleic acid can optionally comprise a packaging signal (e.g., an alphavirus or VEE packaging signal).

A “chimeric” virus as used herein comprises elements from two or more viruses. For example, a chimeric virus can comprise structural proteins from one (or more) viruses and a genomic nucleic acid from another virus. In embodiments of the invention, the chimeric virus is a chimeric alphavirus, e.g., comprising a Sindbis genomic RNA and structural proteins from another alphavirus (e.g., VEE, Girdwood S. A., Ockelbo, and the like). In other embodiments of the invention, the chimeric alphavirus comprises Sindbis alphavirus structural proteins and a genomic RNA from another alphavirus (e.g., VEE, Girdwood S. A., Ockelbo, and the like). Alternatively, a “chimeric virus” comprises structural proteins and/or nucleic acid from two or more viruses, and a “chimeric alphavirus” comprises structural proteins and/or nucleic acid from two or more alphaviruses (e.g., VEE and Sindbis).

An “infectious” virus particle is one that can introduce the viral genomic nucleic acid into a permissive cell, typically by viral transduction. Upon introduction into the target cell, the genomic nucleic acid serves as a template for RNA transcription (i.e., gene expression). The “infectious” virus particle may be “replication-competent” (i.e., can transcribe and replicate the genomic nucleic acid) and “propagation-competent” (i.e., results in a productive infection in which new virus particles are produced). In embodiments of the invention, the “infectious” virus particle is a replicon particle that can introduce the genomic nucleic acid (i.e., replicon) into a host cell, is “replication-competent” to replicate the genomic nucleic acid, but is “propagation-defective” or “propagation-incompetent” in that it is unable to produce new virus particles in the absence of helper sequences that complement the deletions or other mutations in the replicon (i.e., provide the structural proteins that are not provided by the replicon).

A “replicating” or “replication-competent” alphavirus genomic nucleic acid or alphavirus particle refers to the ability to replicate the viral genomic nucleic acid. Generally, a “replication-competent” alphavirus genomic nucleic acid or alphavirus particle will comprise sufficient alphavirus non-structural protein coding sequences (i.e., nsP1 through nsP4 coding sequences) to produce functional alphavirus non-structural proteins.

As used herein, the term “deleted” or “deletion” means either total deletion of the specified segment or the deletion of a sufficient portion of the specified segment to render the segment inoperative or nonfunctional, in accordance with standard usage.

### II. Multivalent Compositions Comprising Norovirus VLPs.

The present invention provides compositions and methods to concurrently induce an immune response (e.g., a protective immune response) against two or more noroviruses (e.g., two, three, four, five, six, seven, eight, nine, ten or more). The two or more noroviruses can be from different strains and/or different genoclusters. In some embodiments, protection is provided against one or more norovirus genoclusters and/or strains not included in the immunogenic composition (i.e., cross-protection), for example, GI.1 and/or GII.4. To date, no effective multivalent norovirus vaccine has been reported that protects against a heterologous norovirus that was not included in the vaccine cocktail. Thus, the present invention responds to the long-term difficulty in the art of providing an immunogenic composition directed against noroviruses in view of the large number of antigenically heterogeneous genoclusters and strains.

In embodiments of the invention, the immunogenic compositions of the invention provide humoral, mucosal and/or cellular immunity against one or more homologous norovirus genoclusters and/or strains included in the immunogenic composition and, optionally, one or more heterologous norovirus genoclusters and/or strains not included in the cocktail.

As one aspect, the invention provides a composition comprising VLPs from two or more genoclusters and/or strains of norovirus (e.g., two, three, four, five, six, seven, eight, nine, ten or more), optionally an immunogenic formulation in a pharmaceutically acceptable carrier. In embodiments of the invention, a norovirus VLP comprises, consists essentially of, or consists of the norovirus major (ORF2) and/or minor (ORFS) capsid proteins. The VLPs can be from contemporary and/or ancestral strains of the norovirus genoclusters.

In embodiments of the invention, the invention comprises a composition comprising two or more nucleic acids (e.g., a plasmid or a viral vector such as an alphavirus vector or baculovirus vector) expressing the two or more different norovirus VLPs, optionally as an immunogenic formulation in a pharmaceutically acceptable carrier. For example, the invention provides a composition comprising two or more alphavirus vectors (e.g., Venezuelan Equine Encephalitis virus), each alphavirus vector expressing a different norovirus VLP. The composition can further comprise nucleic acids encoding other immunogens (e.g., a plasmid or a viral vector such as an alphavirus vector or baculovirus vector expressing an immunogen from one or more different pathogens).

In one embodiment, the compositions of the invention induce humoral, mucosal and/or cellular immunity (optionally, protective immunity) against one or more of the norovirus genoclusters and/or strains included within the immunogenic composition, optionally all of the norovirus genoclusters and/or strains included in the composition.

In one embodiment, the composition induces humoral, mucosal and/or cellular immunity (optionally, protective immunity) against one or more norovirus genoclusters and/or strains (e.g., one, two, three, four, five, six, seven, eight, nine or ten) not included within the immunogenic composition (e.g. the GII.4 genocluster or a GII.4 strain).

The compositions of the invention can include VLPs comprising, consisting essentially of, or consisting of VLPs from one or more of norovirus genogroups GI, GII, GIII, GIV and GV, in any combination. In embodiments of the invention, the composition comprises VLPs comprising, consisting essentially of, or consisting of VLPs from one or more, two or more, or three or more GI and/or GII genogroup noroviruses.

For example, in one embodiment, the composition includes VLPs comprising, consisting essentially of, or consisting of VLPs from one or more GI norovirus genoclusters and/or strains. For example, the composition can include VLPs comprising, consisting essentially of, or consisting of VLPs from one or more, two or more, three or more, four or more, five or more, six or more, seven or more, or all eight of GI.1, GI.2, GI.3, GI.4, GI.5, GI.6, GI.7, GI.8, in any combination thereof. In one embodiment, the composition induces humoral, mucosal and/or cellular immunity against one or more of the GI norovirus genoclusters and/or strains included in the composition. In one embodiment, the composition induces humoral, mucosal and/or cellular immunity against one or more GI norovirus genoclusters and/or strains not included within the immunogenic composition. Further, the composition can optionally induce humoral, mucosal and/or cellular immunity against any other norovirus not included within the immunogenic composition.

As another nonlimiting illustration, the composition can include VLPs comprising, consisting essentially of, or consisting of VLPs from one or more GII norovirus genoclusters and/or strains. For example, the composition can include VLPs comprising, consisting essentially of, or consisting of VLPs from one or more, two or more, three or more, four or more, five or more, six or more, seven or more, eight or more, nine or more, ten or more, eleven or more, twelve or more, thirteen or more, fourteen or more, or all fifteen of GII.1, GII.2, GII.3, GII.4, GII.6, GII.7, GII.8, GII.9, GII.10, GII.11, GII.13, GII.14, GII.17, GII.18 and GII.b, in any combination thereof. In one embodiment, the composition induces humoral, mucosal and/or cellular immunity against one or more of the GII norovirus genoclusters and/or strains included in the composition. In one embodiment, the composition induces humoral, mucosal and/or cellular immunity against one or more GII norovirus genoclusters and/or strains not included within the immunogenic composition. Further, the composition can optionally induce humoral, mucosal and/or cellular immunity against any other norovirus not included within the immunogenic composition.

In representative embodiments, the composition can include VLPs comprising, consisting essentially of, or consisting of a VLP from one or more GI norovirus genoclusters and/or strains and a VLP from one or more GII norovirus genoclusters and/or strains (each as described in the preceding two paragraphs). In one embodiment, the composition induces humoral, mucosal and/or cellular immunity against one or more of the GI and/or GII norovirus genoclusters and/or strains included in the composition. In one embodiment, the composition induces humoral, mucosal and/or cellular immunity against one or more GI norovirus genoclusters and/or strains not included within the immunogenic composition and/or one or more GII norovirus genoclusters and/or strains not included within the immunogenic composition. Further, the composition can optionally induce humoral, mucosal and/or cellular immunity against any other norovirus not included within the immunogenic composition.

Those skilled in the art will appreciate that the VLPs may include further components, for example, that enhance the immunogenicity and/or stability of the VLPs, or that facilitate purification and/or detection of the VLPs.

There are currently 18 GII strains and 8 GI strains. It is understood by those skilled in the art that new strains are constantly evolving and being identified. Further, there will be antigenic drift within strains (e.g., GII.3 and GII.4).

In other representative embodiments, the composition includes VLPs comprising, consisting essentially of or consisting of VLPs from GI.1, GI.2, GI.3, GI.4, GI.5, GI.6, GI.7, GI.8, GII.1, GII.2, GII.3, GII.4, GII.6, GII.7, GII.8, GII.9, GII.10, GII.11, GII.13, GII.14, GII.17, GII.18 and/or GII.b, in any combination.

In embodiments of the invention, the composition includes VLPs comprising, consisting essentially of or consisting of VLPs from GII.1, GII.2, GII.3 and/or GII.4, in any combination. The VLPs can be from contemporary and/or ancestral GI strains. In representative embodiments, the composition includes VLPs comprising, consisting essentially of or consisting of VLPs from GII.4.

In embodiments of the invention, the composition includes VLPs comprising, consisting essentially of or consisting of VLPs from GI.2, GI.3, and/or GI.4, in any combination. The VLPs can be from contemporary and/or ancestral GII strains.

In embodiments of the invention, the composition includes VLPs comprising, consisting essentially of or consisting of VLPs from GI.2, GI.3, GI.4, GII.1, GII.2, GII.3 and GII.4, in any combination. The VLPs can be from contemporary and/or ancestral GI and GII strains.

As another alternative, the composition can include a collection of ancestral and/or contemporary strains from one, two, three or more norovirus genoclusters. For example, the composition can comprise, consist essentially of, or consist of VLPs from two or more, three or more, or four or more GII.4 strains (e.g., from 1987, 1997, 2002 and 2007).

Those skilled in the art will recognize that the mix of genogroups, genoclusters and/or strains included within the composition can be based on diagnostic screening for a representative mix of noroviruses reflecting the current global pandemic strain(s) and/or identification of noroviruses that are currently causing disease in animal (e.g., human) populations.

### III. Adjuvants.

In representative embodiments, the compositions of the invention comprise an adjuvant, optionally only one adjuvant. Alternatively, an adjuvant can be administered concurrently (to have a combined effect, e.g., within hours of each other) with a composition of the invention, but in a separate composition.

The inventors have surprisingly found that a single viral adjuvant can unexpectedly enhance the immune response to two or more (e.g., two, three, four, five, six, seven, eight, nine, ten or more) antigenically distinct norovirus VLPs, further including an enhanced immune response to norovirus genoclusters and/or strains not included in the immunogenic formulation. It is known in the art that the immune response to an antigen is more effectively enhanced by particular adjuvant pathways than others. Thus, it is quite surprising that the present inventors have demonstrated that the immune response to a number of antigenically distinct norovirus VLPs can be enhanced by a single adjuvant and further provide an immune response to heterologous norovirus genoclusters and/or strains not included in the immunogenic formulation.

Nonlimiting examples of adjuvants include an aluminum salt such as aluminum hydroxide gel (alum), aluminum phosphate, or algannmulin, a salt or mineral gels of calcium, magnesium, iron and/or zinc, an insoluble suspension of acylated tyrosine, or acylated sugars, cationically or anionically derivatized polysaccharides, polyphosphazenes, a saponin such as Quil-A, an oil emulsion, such as water-in-oil and water-in-oil-in-water, complete or incomplete Freund's, CpG or any combination of the foregoing. In particular embodiments, the adjuvant is a depot adjuvant.

In representative embodiments of the invention, the adjuvant is a viral adjuvant as described in US Patent Publication No. US 2008/0279891. The viral adjuvant enhances the immune response of a host (e.g., cellular and/or humoral response) against an immunogen that is independent of (e.g., is not presented by or encoded by) the viral adjuvant. In particular embodiments, the viral adjuvant enhances mucosal immunity against the immunogen.

The viral adjuvant can be derived from any suitable virus. In particular embodiments of the invention, the viral adjuvant is an RNA viral adjuvant, i.e., comprises a viral genomic RNA (typically a modified form of a viral genomic RNA) or a DNA molecule that encodes a viral genomic RNA.

The viral adjuvant can be a viral particle adjuvant, which comprises a live, live attenuated, killed and/or chimeric virus particle. Optionally, the viral adjuvant comprises a replicating (i.e., replication-competent) virus particle. In particular embodiments, the viral particle adjuvant is an arbovirus (e.g., a flavivirus, alphavirus or virus in the family Bunyaviridae), a retrovirus, a rotavirus, a coronavirus, an orthomyxovirus, a reovirus, a herpesvirus, a nidovirus, a norovirus, and/or a picornavirus. In other embodiments, the viral adjuvant comprises a virus particle (including replicating virus particles) that uses a mucosal surface for viral entry into the host.

Alternatively, the viral adjuvant comprises components derived from any of the foregoing viruses (e.g., structural proteins and/or nucleic acids, including replicating nucleic acids), optionally in a modified form.

In particular embodiments, the viral adjuvant is an alphavirus adjuvant, more particularly a VEE viral adjuvant. By “alphavirus adjuvant” or “VEE viral adjuvant” it is meant that the viral adjuvant comprises (1) a viral coat comprising one, two or more alphavirus or VEE structural proteins, respectively (e.g., E1, E2 and/or capsid), for example, all of the viral structural proteins in the viral coat can be alphavirus or VEE structural proteins (e.g., E1, E2 and capsid), respectively; and/or (2) an alphavirus or VEE genomic RNA (e.g., a replicating alphavirus or VEE genomic RNA), respectively; and/or (3) a DNA that encodes an alphavirus or VEE genomic RNA, respectively. As described herein, the alphavirus or VEE genomic RNA encompasses modified genomes. In particular embodiments, the alphavirus adjuvant comprises a replicating alphavirus or VEE virus particle, a replicating viral particle comprising an alphavirus or VEE virion coat, or a replicating viral particle comprising an alphavirus or VEE genomic RNA.

Those skilled in the art will appreciate that an alphavirus adjuvant or VEE adjuvant comprising an alphavirus or VEE virion coat, respectively, can further comprise a viral nucleic acid from another virus, either alphavirus or non-alphavirus. Likewise, an alphavirus adjuvant or VEE adjuvant comprising an alphavirus or VEE genomic RNA, respectively, can further comprise a virion coat from another virus, either alphavirus or non-alphavirus.

The viral adjuvant can comprise a wild-type virus, an attenuated live virus and/or an inactivated (i.e., killed) virus. In some embodiments, the viral adjuvant is not an inactivated virus.

As another alternative, the viral adjuvant can comprise a viral genomic nucleic acid or can be a nucleic acid that encodes a nucleic acid derived from viral genomic nucleic acid, for example, as a liposomal formulation. Optionally, the viral nucleic acid is replication-competent.

In some embodiments of the invention, the viral adjuvant comprises structural proteins assembled into a virus-like particle that does not package a genomic nucleic acid or the unassembled viral structural protein(s) (e.g., delivered as a liposomal formulation). To illustrate, the alphavirus E1, E2 glycoproteins and/or the capsid protein, unassembled or assembled as an virus-like particle can be administered, for example, as a liposomal formulation.

In other embodiments, the viral adjuvant can further comprise one or more of the structural proteins (e.g., the alphavirus or VEE E1 and/or E2 glycoproteins) from one of the viruses described above so that the viral adjuvant targets to the same cell(s) as the virus from which the structural protein(s) is derived (e.g., is pseudotyped). In other embodiments, the viral adjuvant comprises a viral nucleic acid (for example, a replicating viral nucleic acid), which for example, can be an alphavirus nucleic acid. In still other embodiments, the viral adjuvant is a chimeric virus in which the structural proteins and/or genomic nucleic acid are derived from different viruses (e.g., two different alphaviruses such as Sindbis and VEE).

In some embodiments of the invention, the viral adjuvant is replication-competent (e.g., a replication-competent virus particle or viral nucleic acid).

In particular embodiments, the viral adjuvant is a propagation-defective virus particle that cannot produce new virus particles upon infection of host cells. According to this embodiment, the viral adjuvant can be replication-competent in that it can infect a host cell and replicate and transcribe the viral genome, but cannot produce new virions (e.g., the virus is a replicon particle). Thus, the adjuvant virus comprises nonstructural protein sequences sufficient to provide replicase and transcriptase functions.

In other embodiments, the viral adjuvant is both propagation and replication-incompetent (e.g., an ultraviolet light or chemically inactivated virus).

The viral adjuvant can be propagation-defective because it is defective for expression of (i.e., is unable to produce a functional form of) at least one or all of the viral structural proteins required to assemble new virus particles (e.g., alphavirus E1, E2 and/or capsid proteins). In other words, the viral adjuvant comprises a modified viral genome or a nucleic acid (that encodes a modified viral genome that is defective for expression of at least one viral structural protein required for production of new virus particles. For example, one or more of the viral structural protein genes can be inactivated by a mutation and/or by deletion. In representative embodiments, the viral adjuvant cannot produce any of the viral structural proteins. In other particular embodiments, the modified viral genome lacks all or essentially all of the sequences encoding the viral structural proteins.

Additionally or alternatively, in other embodiments, the genomic promoter that drives expression of the viral structural protein genes (e.g., the alphavirus 26S promoter) is inactivated (e.g., so that no detectable promoter activity is observed, for example, by measuring RNA transcription or protein expression driven by the promoter) or partially or completely deleted such that the promoter is absent or non-functional. Inactivating mutations can comprise insertions, substitutions and/or deletions. To illustrate, the promoter can be inactivated by mutation of cis-acting sequences, for example, by mutagenesis of sequence elements within the promoter region that are required for binding to the RNA polymerase complex. Alternatively or additionally, the viral polymerase can be mutated, e.g., mutation of the viral polymerase encoded by the alphavirus nsP4 gene so that it no longer recognizes the 26S promoter. Further, specific mutations in the alphavirus nsP1-nsP3 proteins are associated with loss of subgenomic RNA synthesis (while retaining genomic RNA synthesis). Such mutations can be incorporated into the viral adjuvants of the invention to render the viral adjuvant defective for subgenomic RNA synthesis as well as production of new virus particles.

In particular embodiments, the minimal VEE 26S promoter region from −19 to +5 can be deleted (numbering with reference to the transcriptional start site for the 26S subgenomic promoter), which corresponds to 7513 to 7536 of the published sequence of the Trinidad donkey strain of the VEE genome (GenBank Accession No. J04332). In some embodiments, the deletion can be extended further, e.g., to nucleotide −30, −40, −50, −75 or −100 with respect to the transcriptional start site. Optionally, the deletion can extend beyond the minimal promoter in the 5′ and/or 3′ direction. In particular embodiments, the deletion does not extend into the nsp4 coding sequence. Alternatively, the promoter can be inactivated by deleting portions of the VEE 26S promoter (e.g., at least about three, four, five, six, eight, ten, twelve, fifteen or more nucleotides of the minimal promoter region from −19 to +5 or the broader promoter region from about −100 to +5), which can optionally include (i.e., span) the transcriptional start site. In specific embodiments, the deletion is from −5 through to +5 or even further downstream with respect to the transcriptional start site.

Inactivating mutations in the Sindbis virus 26S promoter have' been described in U.S. Pat. No. 6,376,236.

In particular embodiments, the inactivating mutations or deletions in the alphavirus 26S promoter do not result in substitutions, insertions and/or deletions in the alphavirus nsp4 coding sequence. In other embodiments, any mutation(s) in the nsp4 coding sequence is a silent mutation that does not result in an amino acid substitution, insertion or deletion. As a further alternative, in some embodiments, there are one, two, three or more amino acid substitutions and/or one, two, three or more amino acid insertions and/or a deletion (including truncation) of one, two or three or more amino acids in the nsP4 protein, which mutations do not unduly decrease (for example, less than about a 5%, 10%, 15%, 20%, 25% or 35% decrease) the activity of the nsP4 protein (e.g., the polymerase activity, for example, as determined by ability of the modified alphavirus genome to self-replicate). For example, in some embodiments, an “undue” decrease in nsP4 activity would be a decrease that results in a substantial decrease in adjuvant activity.

In representative embodiments of the invention, the promoter that drives expression of the structural proteins (e.g., alphavirus 26S promoter) is modified so that it has reduced activity (e.g., transcriptional activity), for example, by at least about 20%, 25%, 35%, 50%, 60%, 75%, 80%, 85%, 90%, 95%, 97%, 98%, 99% or more). Reductions in transcriptional activity can be determined by any method known in the art including methods that measure RNA levels and those that measure protein expression. Alternatively, the promoter can have reduced activity in driving protein expression due to a change at the transcriptional, translational or post-translational level or any other mechanism that results in reduced protein expression. In particular embodiments, the promoter sequence is modified by insertion, substitution and/or deletion to reduce the activity (e.g., transcriptional activity) of the promoter. For example, any one, two, three, four, five, six, seven, eight, nine, ten, eleven, twelve, thirteen, fourteen, fifteen or more nucleotides can be deleted from the promoter. Substitution mutations can be made at any one, two, three, four, five, six, seven, eight, nine, ten or more nucleotide positions within the promoter region. As another possibility, insertions of one, two, three, four, five, six, seven, eight, nine or ten nucleotides or more can be made at one or more sites (e.g., two, three, four, five or six sites) within the promoter region.

As one example, the modified viral genome can be a modified alphavirus genome comprising a mutated 26S subgenomic promoter that has reduced transcriptional activity (e.g., ability to drive expression of a sub-genomic transcript) and/or reduced activity in driving protein expression. In particular embodiments, there are no mutations in the alphavirus nsp4 coding sequence. In other embodiments, any mutation(s) in the nsp4 coding sequence is a silent mutation that does not result in an amino acid substitution, insertion or deletion. As a further alternative, in some embodiments, there are one, two, three or more amino acid substitutions and/or one, two, three or more amino acid insertions and/or a deletion (including truncation) of one, two or three amino acids or more in the nsP4 protein, which mutations do not unduly decrease (for example, less than a 5%, 10%, 15%, 20%, 25% or 35% decrease) the activity of the nsP4 protein (e.g., polymerase activity, for example, as determined by ability of the modified alphavirus genome to replicate). For example, in some embodiments, an “undue” decrease in nsP4 activity would be a decrease that results in a substantial decrease in adjuvant activity.

The minimal VEE 26S promoter is from −19 to +5 with respect to the transcriptional start site (see, e.g., published sequence of the Trinidad donkey strain of the VEE genome; GenBank Accession No. J04332), although the promoter extends beyond this minimal region to approximately nucleotide −100 or even further in the upstream direction. In particular embodiments, the VEE promoter sequence is modified by insertion, substitution and/or deletion to reduce the activity (e.g., transcriptional activity) of the promoter. For example, any one, two, three, four, five, six, seven, eight, nine, ten, eleven, twelve, thirteen, fourteen, fifteen or more nucleotides can be deleted from the promoter. Substitution mutations can be made at any one, two, three, four, five, six, seven, eight, nine, ten or more nucleotide positions within the promoter region. As another possibility, insertions of one, two, three, four, five, six, seven, eight, nine or ten nucleotides or more can be made at one or more sites (e.g., two, three, four, five or six sites) within the promoter region, e.g., between positions −19/−18, positions −18/−17, positions −17/−16, positions −16/−15, positions −15/−14, positions −14/−13, positions −13/−12, positions −12/−11, positions −11/−10, positions −10/−9, positions −9/−8, positions −8/−7, positions, −7/−6, positions −6/−5, positions −5/−4, positions −4/−3, positions −3/−2, positions −2/−1, positions −1/+1, positions +1/+2, positions +2/+3, positions +3/+4 and/or positions +4/+5 (numbering with respect to the transcriptional start site). As a further option, insertions, substitutions and/or deletions (including truncations) can be made in (or extend into) the promoter region further upstream of the transcriptional start site (e.g., from about nucleotide −100 to +5).

While not wishing to be held to any particular mechanism of action, it appears that the inclusion of a 26S promoter with reduced activity (e.g., transcriptional activity) results in increased cell death and cytokine (e.g., interferon) production, which are believed to enhance adjuvant activity. Reduced cell viability and enhanced induction of cytokines may be attributable, at least in part, to elevated levels of alphavirus genomic transcripts and/or an increased ratio of genomic to subgenomic transcripts in the host cell. Thus, in particular embodiments, the modified 26S promoter has reduced activity (e.g., transcriptional activity) and results in enhanced death of host cells and/or enhanced cytokine (e.g., interferon) production.

In particular embodiments, modified VEE 26S promoters having reduced activity (e.g., transcriptional activity) comprise a 3 (e.g., CAG), 4 (e.g., TCAG) or 5 (e.g., GTCAG) nucleotide insertion between positions −5/−4 (corresponding to nucleotides 7527 and 7528 with reference to GenBank Accession No. J04322).

Other exemplary modified VEE 26S promoters comprise point mutations at nucleotide 7505 (e.g., C to G) and/or 7517 (e.g., C to G) (numbering is with reference to GenBank Accession No. J04322), which corresponds to nucleotide positions −27 and −15, respectively, with reference to the transcriptional start site. In particular embodiments, the mutations are silent mutations.

In particular embodiments, there are no mutations in the VEE nsp4 coding sequence. In other embodiments, any mutation(s) in the nsp4 coding sequence is a silent mutation that does not result in an amino acid substitution, insertion and/or deletion. As a further alternative, in some embodiments, there are one, two, three or more amino acid substitutions and/or one, two, three or more amino acid insertions and/or a deletion (including truncation) of one, two or three amino acids or more in the nsP4 protein, which mutations do not unduly decrease (for example, less than a 5%, 10%, 15%, 20%, 25% or 35% decrease) the activity of the nsP4 protein (e.g., as determined by ability of the modified alphavirus genome to replicate). For example, in some embodiments, an “undue” decrease in nsP4 activity would be a decrease that results in a substantial decrease in adjuvant activity.

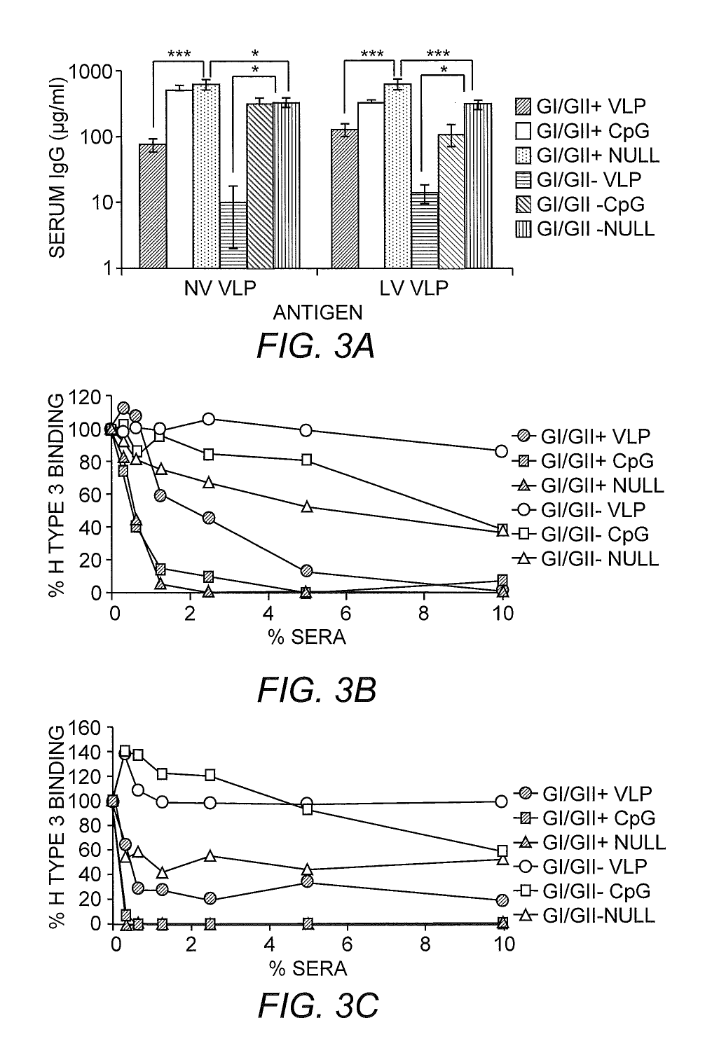

Mutations are known in the art that reduce the transcriptional activity of the Sindbis virus 26S promoter. For example, Groukoui et al. describes a 3 nucleotide insertion (GUC) mutation between positions −5/−4 of the transcriptional start site of Sindbis virus that substantially reduces subgenomic RNA synthesis (Groukoui et al., (1989) J. Virology 63:5216-5227). This mutation also resulted in an arginine insertion between residues 608 and 609 of the nsP4 protein. Further, U.S. Pat. No. 6,376,236 describes a minimal Sindbis promoter from nucleotides 7579 to 7612 (see full length sequence in FIG. 3 of this patent), and describes modified Sindbis 26S promoters having reduced transcriptional activity.

Other modifications can be made to the Sindbis virus 26S promoter sequence to reduce the activity (e.g., transcriptional activity) thereof, e.g., by insertion, substitution and/or deletion to reduce the activity (e.g., transcriptional activity) of the promoter. For example, any one, two, three, four, five, six, seven, eight, nine, ten, eleven, twelve, thirteen, fourteen, fifteen or more nucleotides can be deleted from the promoter. Substitution mutations can be made at any one, two, three, four, five, six, seven, eight, nine, ten or more nucleotide positions within the promoter region. As another possibility, insertions of one, two, three, four, five, six, seven, eight, nine or ten nucleotides or more can be made at one or more sites (e.g., two, three, four, five or six sites) within the promoter region, e.g., between positions −19/−18, positions −18/−17, positions −17/−16, positions −16/−15, positions −15/−14, positions −14/−13, positions −13/−12, positions −12/−11, positions −11/−10, positions −10/−9, positions −9/−8, positions −8/−7, positions, −7/−6, positions −6/−5, positions −5/−4, positions −4/−3, positions −3/−2, positions −2/−1, positions −1/+1, and/or positions +1/+2 (numbering with respect to the transcriptional start site). As a further option, insertions, substitutions and/or deletions (including truncations) can be made in (or extend into) the promoter region further upstream of the transcriptional start site (e.g., from about nucleotide −100 to +5).

In particular embodiments, there are no mutations in the Sindbis virus nsp4 coding sequence. In other embodiments, any mutation(s) in the nsp4 coding sequence is a silent mutation that does not result in an amino acid substitution, insertion or deletion. As a further alternative, in some embodiments, there are one, two, three or more amino acid substitutions and/or one, two, three or more amino acid insertions and/or a deletion (including truncation) of one, two or three amino acids or more in the Sindbis virus nsP4 protein, which mutations do not unduly decrease (for example, less than a 5%, 10%, 15%, 20%, 25% or 35% decrease) the activity of the nsP4 protein (e.g., polymerase activity, for example, as determined by ability of the modified alphavirus genome to replicate). For example, in some embodiments, an “undue” decrease in nsP4 activity would be a decrease that results in a substantial decrease in adjuvant activity.

One skilled in the art can make corresponding mutations to those described above to reduce the activity (e.g., transcriptional activity) of 26S promoters from other alphaviruses (e.g., Semliki Forest Virus, Girdwood virus, etc.).

In some embodiments, the alphavirus genome comprises two or more 26S promoter sequences, one or both of which may be inactivated and/or modified to have reduced activity (e.g., transcriptional activity).

As described above, the viral adjuvant does not express the norovirus VLP, i.e., the genome of the adjuvant virus does not comprise a heterologous nucleic acid sequence that encodes the norovirus VLP. In embodiments of the invention, the viral adjuvant does not present or express any norovirus immunogen, i.e., the immunogen is not presented as part of the virion structure and the genome of the adjuvant virus does not comprise a heterologous nucleic acid sequence that encodes the immunogen.

However, it will be appreciated by those skilled in the art that the viral adjuvant may express one or more different antigens or an untranslated RNA.

In particular embodiments, the adjuvant virus expresses a polypeptide of interest including but not limited to another immunogen (i.e., other than the norovirus VLP), a reporter protein (e.g., an enzyme) and/or an immunomodulatory polypeptide such as a cytokine or chemokine (e.g., α-interferon, β-interferon, γ-interferon, ω-interferon, τ-interferon, interleukin-1α, interleukin-1β, interleukin-2, interleukin-3, interleukin-4, interleukin 5, interleukin-6, interleukin-7, interleukin-8, interleukin-9, interleukin-10, interleukin-11, interleukin 12, interleukin-13, interleukin-14, interleukin-18, B cell Growth factor, CD40 Ligand, tumor necrosis factor-α, tumor necrosis factor-β, monocyte chemoattractant protein-1, granulocyte-macrophage colony stimulating factor, lymphotoxin, CCL25 [MECK], and CCL28 [TECH]). Alternatively, the viral adjuvant expresses a functional untranslated RNA.

Reporter proteins are known in the art and include, but are not limited to, Green Fluorescent Protein, β-galactosidase, alkaline phosphatase, chloramphenicol acetyltransferase, and the like.

In some embodiments of the invention, the viral adjuvant comprises a “stuffer” nucleic acid, typically a “spacer” inserted in place of deleted structural protein coding sequences. The stuffer nucleic acid does not encode a polypeptide of interest or functional untranslated RNA, and is inserted into the genome to maintain the size of the genome in the range preferred by the virus (e.g., because of deletion of one or more of the viral structural protein genes). In other embodiments, the viral adjuvant comprises any other nucleic acid that is transcribed and optionally translated, but does not encode the immunogen.

In particular embodiments, the viral adjuvant does not comprise a heterologous nucleic acid that encodes a polypeptide of interest and/or functional untranslated RNA (i.e., the virus does not express a heterologous or foreign polypeptide of interest and/or functional untranslated RNA). In other words, the viral adjuvant does not comprise a foreign sequence that encodes a polypeptide of interest and/or functional untranslated RNA.

In other representative embodiments, the viral adjuvant is an “empty” virus particle or genomic nucleic acid that does not comprise a heterologous nucleic acid sequence (e.g., in place of deleted structural protein coding sequences). Those skilled in the art will appreciate that by “heterologous nucleic acid” as used in this context it is intended a nucleic acid that is foreign or exogenous to the virus and which is transcribed, and optionally translated, to produce a polypeptide of interest or functional untranslated RNA of interest or a “stuffer” nucleic acid as described above. Thus, it will further be recognized by those skilled in the art that the phrase “does not comprise a heterologous nucleic acid sequence” does not exclude the presence of all other foreign sequences in the virus, for example, foreign promoter sequences, attenuating mutations, mutations or foreign sequences that affect virus tropism, immunogenicity or virus clearance and/or other modifications that are introduced, for example, to alter pathogenesis, replication, transcription and/or translation. Further, there may be residual sequences, both native and foreign, (e.g., as a result of the experimental procedures used to produce the construct, for example, restriction sites) in the construct that may be transcribed or even translated (e.g., if operably associated with the alphavirus 26S promoter). Such sequences, however, do not encode a polypeptide of interest or functional untranslated RNA, as those terms are used herein.

In representative embodiments, the viral adjuvant comprises a viral genomic nucleic acid that lacks sequences encoding one or more, optionally all, of the viral structural proteins and further in which the viral promoter that is operably associated therewith is inactivated, partially or completely deleted therefrom, or is otherwise modified to reduce the activity (e.g., transcriptional activity) of the promoter. For example, the promoter and nonstructural protein coding sequences from nucleotide 7562 (numbering with respect to the published sequence of the Trinidad donkey strain of the VEE genome; GenBank accession #J04332), corresponding to −6 with respect to the transcriptional start site through the structural protein coding sequences are deleted. Optionally, the adjuvant virus does not comprise a heterologous nucleic acid sequence (as described herein). Thus, according to this embodiment, the viral adjuvant can be a “minimal” replication-competent nucleic acid or viral particle that lacks sequences encoding the structural proteins (i.e., is propagation incompetent) and the genomic promoter associated therewith, but does not comprise a heterologous nucleic acid in the form of a sequence that encodes a polypeptide of interest or functional untranslated RNA or a stuffer RNA. In some embodiments, the “minimal” nucleic acid or virus particle comprises sequences necessary for the nucleic acid or virus particle to self-replicate. Alternatively, the viral adjuvant can be replication-competent nucleic acid or viral particle in which the promoter driving expression of the structural proteins has been modified to reduce the activity (e.g., transcriptional activity) of the promoter (e.g., an alphavirus 26S promoter) and further the viral adjuvant does not comprise a heterologous nucleic acid in the form of a sequence that encodes a polypeptide of interest or functional untranslated RNA or a stuffer RNA. In some embodiments, this modified nucleic acid or virus particle comprises sequences necessary for the nucleic acid or virus particle to self-replicate.

In illustrative embodiments of the invention, the viral adjuvant comprises: (a) a modified viral genome that lacks sequences encoding the viral structural proteins required for production of new virus particles; wherein the modified viral genome does not comprise a heterologous nucleic acid sequence that encodes a polypeptide of interest or a functional untranslated RNA, and optionally (b) a viral coat comprising virus structural proteins. In other embodiments, the viral adjuvant comprises a nucleic acid molecule(s) (e.g., DNA and/or RNA) that encodes the modified viral genome and, optionally, the viral coat. In particular embodiments, the viral adjuvant comprises one, two or more alphavirus structural proteins (e.g., all of the structural proteins in the virion coat are alphavirus structural proteins). In other embodiments, the modified viral genome is a modified alphavirus genome, optionally packaged within viral structural proteins (e.g., alphavirus structural proteins). Optionally, the 26S promoter is inactivated, partially or entirely deleted and/or the 26S promoter has been modified to reduce activity (e.g., transcriptional activity) of the promoter. Alternatively, the viral adjuvant comprises a nucleic acid (e.g., DNA and/or RNA) that encodes the modified alphavirus genome. In some embodiments, the viral adjuvant is a self-replicating viral adjuvant.

Further, the viral adjuvant can be a VEE viral adjuvant comprising a virion coat comprising one, two or more VEE structural proteins (e.g., all of the structural proteins in the virion coat are VEE structural proteins). In other particular embodiments, the viral adjuvant is a VEE viral adjuvant comprising a modified VEE genome that lacks the sequences encoding the VEE structural proteins required for production of new virus particles. Optionally, the VEE viral adjuvant comprises a modified viral genome that lacks sequences encoding the viral structural proteins. In particular embodiments, the modified viral genome does not comprise a heterologous nucleic acid sequence that encodes a polypeptide of interest or functional untranslated RNA. In representative embodiments, the viral adjuvant comprises a modified VEE genomic nucleic acid (as described above), packaged within a virion coat (also as described above, for example, a virion coat of VEE structural proteins). Alternatively, the VEE viral adjuvant comprises a nucleic acid(s) (e.g., DNA and/or RNA) that encodes the modified VEE genome and, optionally, viral structural proteins. In particular embodiments, the VEE 26S promoter is inactivated, partially or entirely deleted and/or the 26S promoter has been modified to reduce activity (e.g., transcriptional activity) of the promoter. In some embodiments, the VEE viral adjuvant is a self-replicating VEE viral adjuvant.

In other embodiments, the viral adjuvant is a VEE viral adjuvant comprising: VEE structural proteins; and a modified VEE genome that lacks the genes encoding the VEE structural proteins required for production of new virus particles.

In some embodiments, VEE viral adjuvants comprise a modified VEE genome comprising an attenuating mutation at nucleotide 3 of the genome (e.g., the mutation can be a G→A, U or C, but is preferably a G→A mutation), which mutation has been observed to enhance cytokine production in host cells. Attenuating mutations are discussed in more detail hereinbelow.

A. Alphavirus Adjuvants.

The present invention may be practiced using alphavirus adjuvants, for example, a propagation-incompetent, replicating, alphavirus adjuvant such as an alphavirus replicon vector (as described below), an alphavirus-like particle of assembled structural proteins, or an alphavirus nucleic acid. Alphavirus vectors, including replicon vectors, are described in U.S. Pat. No. 5,505,947 to Johnston et al.; U.S. Pat. No. 5,792,462 to Johnston et al.; U.S. Pat. No. 6,156,558; U.S. Pat. No. 6,521,325; U.S. Pat. No. 6,531,135; U.S. Pat. No. 6,541,010; and Pushko et al. (1997) Virol. 239:389-401; U.S. Pat. No. 5,814,482 to Dubensky et al.; U.S. Pat. No. 5,843,723 to Dubensky et al.; U.S. Pat. No. 5,789,245 to Dubensky et al.; U.S. Pat. No. 5,739,026 to Garoff et al. In embodiments of the invention, the alphavirus vector is a Sindbis (e.g., TR339) or VEE vector, a Sindbis or VEE replicon vector, a Sindbis chimeric vector comprising a Sindbis genomic RNA or Sindbis glycoproteins (i.e., E1 and E2), or a VEE chimeric vector comprising a VEE genomic RNA or VEE glycoproteins (Le., E1 and E2).

The alphavirus adjuvants employed in the present invention may be a chimeric alphavirus particle, as that term is understood in the art and defined herein. For example, the alphavirus structural proteins may be from one alphavirus (e.g., VEE or a Sindbis virus such as TR339) and a genomic RNA packaged within the virion may be from another alphavirus. Alternatively, the alphavirus coat can be assembled from structural proteins derived from more than one alphavirus.

i. Double Promoter Vectors.

In embodiments of the invention, the viral adjuvant comprises an alphavirus double promoter vector (e.g., a viral particle or a naked genomic RNA or a nucleic acid such as a DNA encoding the genomic RNA). A double promoter vector is typically a replication and propagation competent virus that retains the sequences encoding the alphavirus structural proteins sufficient to produce an alphavirus particle. Double promoter vectors are described in U.S. Pat. Nos. 5,185,440, 5,505,947 and 5,639,650. Illustrative alphaviruses for constructing the double promoter vectors are Sindbis (e.g., TR339), Girdwood and VEE viruses. In addition, the double promoter vector may contain one or more attenuating mutations. Attenuating mutations are described in more detail herein.

In representative embodiments, the double promoter vector is constructed so as to contain a second subgenomic promoter (Le., 26S promoter) inserted 3′ to the viral RNA encoding the structural proteins or between nsP4 and the native 26S promoter. The heterologous RNA may be inserted between the second subgenomic promoter, so as to be operatively associated therewith, and the 3′ UTR of the virus genome. Heterologous RNA sequences of less than about 3 kilobases, less than about 2 kilobases, or less than about 1 kilobase, can be inserted into the double promoter vector. In a representative embodiment of the invention, the double promoter vector is derived from a Sindbis (e.g., TR339) genomic RNA, and the second subgenomic promoter is a duplicate of the Sindbis (e.g., TR339) subgenomic promoter. In an alternate embodiment, the double promoter vector is derived from a VEE genomic RNA (e.g., having a mutation at nt3 of the genomic RNA), and the second subgenomic promoter is a duplicate of the VEE subgenomic promoter.

ii. Replicon Vectors.

The viral adjuvant can comprise an alphavirus replicon vector (e.g., a viral particle or naked genomic RNA or a nucleic acid such as a DNA encoding a genomic RNA), which are infectious, propagation-defective, replicating virus vectors. Replicon vectors are described in more detail in WO 96/37616 to Johnston et al.; U.S. Pat. No. 5,505,947 to Johnston et al.; U.S. Pat. No. 5,792,462 to Johnston et al.; U.S. Pat. No. 6,156,558; U.S. Pat. No. 6,521,325; U.S. Pat. No. 6,531,135; U.S. Pat. No. 6,541,010; and Pushko et al. (1997) Virol. 239:389-401. Illustrative alphaviruses for constructing the replicon vectors according to the present invention are Sindbis (e.g., TR339), Girdwood, VEE, and chimeras thereof.

In general, in the replicon system, the viral genome contains the viral sequences necessary for viral replication (e.g., the nsp1-4 genes), but is modified so that it is defective for expression of at least one viral structural protein required for production of new viral particles. RNA transcribed from this vector contains sufficient viral sequences (e.g., the viral nonstructural genes) responsible for RNA replication and transcription. Thus, if the transcribed RNA is introduced into susceptible cells, it will be replicated and translated to give the replication proteins. These proteins will transcribe the recombinant genomic RNA, and optionally a transgene (if present). The autonomously replicating RNA (i.e., replicon) can only be packaged into virus particles if the defective or alphavirus structural protein genes that are deleted from or defective in the replicon are provided on one or more helper molecules, which are provided to the helper cell, or by a stably transformed packaging cell.

In some embodiments, the helper molecules do not contain the viral nonstructural genes for replication, but these functions are provided in trans by the replicon molecule. The transcriptase functions translated from the replicon molecule transcribe the structural protein genes on the helper molecule, resulting in the synthesis of viral structural proteins and packaging of the replicon into virus-like particles. Optionally, the helper molecules do not contain a functional alphavirus packaging signal. As the alphavirus packaging or encapsidation signal is located within the nonstructural genes, the absence of these sequences in the helper molecules precludes their incorporation into virus particles.

Accordingly, the replicon molecule is “propagation defective” or “propagation incompetent,” as described hereinabove. Typically, the resulting alphavirus particles are propagation defective inasmuch as the replicon RNA in these particles does not encode all of the alphavirus structural proteins required for encapsidation, at least a portion of at least one of the required structural proteins being deleted therefrom, such that the replicon RNA initiates only an abortive infection; no new viral particles are produced, and there is no spread of the infection to other cells. Alternatively, the replicon RNA may comprise one or more mutations within the structural protein coding sequences or promoter driving expression of the structural protein coding sequences, which interfere(s) with the production of a functional structural protein(s).

Typically, the replicon molecule comprises an alphavirus packaging signal.

The replicon molecule is self-replicating. Accordingly, the replicon molecule comprises sufficient coding sequences for the alphavirus nonstructural polyprotein so as to support self-replication. In embodiments of the invention, the replicon encodes the alphavirus nsP1, nsP2, nsP3 and nsP4 proteins.

The replicon molecules of the invention do not encode one or more of the capsid, E1 or E2 alphavirus structural proteins. By “do(es) not encode” one or more structural proteins, it is intended that the replicon molecule does not encode a functional form of the one or more structural proteins and, thus, a complementing sequence must be provided by a helper or packaging cell to produce new virus particles. In embodiments of the invention, the replicon molecule does not encode any of the alphavirus structural proteins.

The replicon may not encode the structural protein(s) because the coding sequence is partially or entirely deleted from the replicon molecule. Alternatively, the coding sequence is otherwise mutated so that the replicon does not express the functional protein. In embodiments of the invention, the replicon lacks all or substantially all of the coding sequence of the structural protein(s) that is not encoded by the replicon, e.g., so as to minimize recombination events with the helper sequences.

In particular embodiments, the replicon molecule may encode at least one, but not all, of the alphavirus structural proteins. For example, the alphavirus capsid protein may be encoded by the replicon molecule. Alternatively, one or both of the alphavirus glycoproteins may be encoded by the replicon molecule. As a further alternative, the replicon may encode the capsid protein and either the E1 or E2 glycoprotein.

In other embodiments, none of the alphavirus structural proteins are encoded by the replicon molecule. For example, all or substantially all of the sequences encoding the structural proteins (e.g., E1, E2 and capsid) may be deleted from the replicon molecule.

In some aspects of the invention, a composition comprising a population of replicon particles of the invention contains no detectable propagation-competent alphavirus particles. Propagation-competent virus may be detected by any method known in the art, e.g., by neurovirulence following intracerebral injection into suckling mice or by passage twice on alphavirus-permissive cells (e.g., BHK cells) and evaluation for virus induced cytopathic effects.

Replicon vectors that do not encode the alphavirus capsid protein, may nonetheless comprise a capsid translational enhancer region operably associated with a heterologous sequence, or the sequences encoding the non-structural proteins and/or encoding the alphavirus structural proteins (e.g., E1 and/or E2 glycoproteins) so as to enhance expression thereof. See, e.g., PCT Application No. PCT/US01/27644; U.S. Pat. No. 6,224,879 to Sjoberg et al., Smerdou et al., (1999) J. Virol. 73:1092; Frolov et al., (1996) J. Virol. 70:1182; and Heise et al. (2000) J. Virol. 74:9294-9299.

In particular embodiments, the replicon vector is an “empty” replicon vector that does not comprise a heterologous nucleic acid sequence (as described herein) or a “minimal” replicon vector in which the 26S subgenomic promoter is deleted or inactivated (also as described herein).

iii. Attenuating Mutations.

The methods of the present invention may also be carried out with alphavirus genomic RNA, structural proteins, and particles including attenuating mutations. The phrases “attenuating mutation” and “attenuating amino acid,” as used herein, mean a nucleotide sequence containing a mutation, or an amino acid encoded by a nucleotide sequence containing a mutation, which mutation results in a decreased probability of causing disease in its host (i.e., reduction in virulence), in accordance with standard terminology in the art. See, e.g., B. Davis et al., MICROBIOLOGY 132 (3d ed. 1980). The phrase “attenuating mutation” excludes mutations or combinations of mutations that would be lethal to the virus.

Appropriate attenuating mutations will be dependent upon the alphavirus used, and will be known to those skilled in the art. Exemplary attenuating mutations include, but are not limited to, those described in U.S. Pat. No. 5,505,947 to Johnston et al., U.S. Pat. No. 5,185,440 to Johnston et al., U.S. Pat. No. 5,643,576 to Davis et al., U.S. Pat. No. 5,792,462 to Johnston et al., and U.S. Pat. No. 5,639,650 to Johnston et al.

When the alphavirus structural proteins are from VEE, suitable attenuating mutations may be selected from the group consisting of codons at E2 amino acid position 76 which specify an attenuating amino acid, preferably lysine, arginine, or histidine as E2 amino acid 76; codons at E2 amino acid position 120 which specify an attenuating amino acid, preferably lysine as E2 amino acid 120; codons at E2 amino acid position 209 which specify an attenuating amino acid, preferably lysine, arginine or histidine as E2 amino acid 209; codons at E1 amino acid 272 which specify an attenuating amino acid, preferably threonine or serine as E1 amino acid 272; codons at E1 amino acid 81 which specify an attenuating amino acid, preferably isoleucine or leucine as E1 amino acid 81; codons at E1 amino acid 253 which specify an attenuating amino acid, preferably serine or threonine as E1 amino acid 253; or the deletion of E3 amino acids 56-59, or a combination of the deletion of E3 amino acids 56-59 together with codons at E1 amino acid 253 which specify an attenuating mutation, as provided above.

Another suitable attenuating mutation is an attenuating mutation at nucleotide 3 of the VEE genomic RNA, i.e., the third nucleotide following the 5′ methylated cap (see, e.g., U.S. Pat. No. 5,643,576 describing a G→C mutation at nt 3). The mutation may be a G→A, U or C, but is preferably a G→A mutation.

When the alphavirus structural and/or non-structural proteins are from S.A.AR86, exemplary attenuating mutations in the structural and non-structural proteins include, but are not limited to, codons at nsP1 amino acid position 538 which specify an attenuating amino acid, preferably isoleucine as nsP1 amino acid 538; codons at E2 amino acid position 304 which specify an attenuating amino acid, preferably threonine as E2 amino acid 304; codons at E2 amino acid position 314 which specify an attenuating amino acid, preferably lysine as E2 amino acid 314; codons at E2 amino acid 372 which specify an attenuating amino acid, preferably leucine, at E2 amino acid residue 372; codons at E2 amino acid position 376 which specify an attenuating amino acid, preferably alanine as E2 amino acid 376; in combination, codons at E2 amino acid residues 304, 314, 372 and 376 which specify attenuating amino acids, as described above; codons at nsP2 amino acid position 96 which specify an attenuating amino acid, preferably glycine as nsP2 amino acid 96; and codons at nsP2 amino acid position 372 which specify an attenuating amino acid, preferably valine as nsP2 amino acid 372; in combination, codons at nsP2 amino acid residues 96 and 372 which encode attenuating amino acids at nsP2 amino acid residues 96 and 372, as described above; codons at nsP2 amino acid residue 529 which specify an attenuating amino acid, preferably leucine, at nsP2 amino acid residue 529; codons at nsP2 amino acid residue 571 which specify an attenuating amino acid, preferably asparagine, at nsP2 amino acid residue 571; codons at nsP2 amino acid residue 682 which specify an attenuating amino acid, preferably arginine, at nsP2 amino acid residue 682; codons at nsP2 amino acid residue 804 which specify an attenuating amino acid, preferably arginine, at nsP2 amino acid residue 804; codons at nsp3 amino acid residue 22 which specify an attenuating amino acid, preferably arginine, at nsP3 amino acid residue 22; and in combination, codons at nsP2 amino acid residues 529, 571, 682 and 804 and at nsP3 amino acid residue 22 which specify attenuating amino acids, as described above.

Other illustrative attenuating mutations include those described in PCT Application No. PCT/US01/27644. For example, the attenuating mutation may be an attenuating mutation at amino acid position 537 of the S.A.AR86 nsP3 protein, for example, a substitution mutation at this position, or a nonsense mutation that results in substitution of a termination codon. Translational termination (i.e., stop) codons are known in the art, and include the “opal” (UGA), “amber” (UAG) and “ochre” (UAA) termination codons. In embodiments of the invention, the attenuating mutation results in a Cys→opal substitution at S.A.AR85 nsP3 amino acid position 537.

Further exemplary attenuating mutations include an attenuating insertion mutation following amino acid 385 of the S.A.AR86 nsP3 protein. In embodiments of the invention, the insertion comprises an insertion of at least about 2, 4, 6, 8, 10, 12, 14, 16 or 20 amino acids. In embodiments of the invention, the inserted amino acid sequence is rich in serine and threonine residues (e.g., comprises at least 2, 4, 6, or 8 such sites) that serve as a substrate for phosphorylation by serine/threonine kinases.

In some embodiments, the attenuating mutation comprises an insertion of the amino acid sequence Ile-Thr-Ser-Met-Asp-Ser-Trp-Ser-Ser-Gly-Pro-Ser-Ser-Leu-Glu-Ile-Val-Asp (SEQ ID NO:1) following amino acid 385 of nsP3 (i.e., the first amino acid is designated as amino acid 386 in nsP3) of S.A.AR86. In other embodiments of the invention, the insertion mutation comprises insertion of a fragment of SEQ ID NO:1 that results in an attenuated phenotype. For example, the fragment can comprise at least about 4, 6, 8, 10, 12,14 or 16 contiguous amino acids from SEQ ID NO:1.

Those skilled in the art will appreciate that other attenuating insertion sequences comprising a fragment of the sequence set forth above, or which incorporate conservative amino acid substitutions into the sequence set forth above, may be routinely identified by those of ordinary skill in the art (as described herein). While not wishing to be bound by any theory, it appears that the insertion sequence of SEQ ID NO:1 is highly phosphorylated at serine residues, which confers an attenuated phenotype. Thus, other attenuating insertion sequences which serve as substrates for serine (or threonine) phosphorylation may be identified by conventional techniques known to those skilled in the art.

Alternatively, or additionally, the attenuating mutation comprises a Tyr→Ser substitution at amino acid 385 of the S.A.AR86 nsP3 (i.e., just prior to the insertion sequence above). This sequence is conserved in the non-virulent Sindbis-group viruses, but is deleted from S.A.AR86.

Other attenuating mutations for S.A.AR86 include attenuating mutations at those positions that diverge between S.A.AR86 and non-neurovirulent Sindbis group viruses, including attenuating mutations at nsP2 amino acid position 256 (e.g., Arg→Ala), 648 (e.g., Ile→Val) or 651 (e.g., Lys→Glu), attenuating mutations at nsP3 amino acid position 344 (e.g., Gly→Glu), 441 (e.g., Asp→Gly) or 445 (e.g., Ile→Met), attenuating mutations at E2 amino acid position 243 (e.g., Ser→Leu), attenuating mutations at 6K amino acid position 30 (e.g., Val→Ile), and attenuating mutations at E1 amino acid positions 112 (e.g., Val→Ala) or 169 (e.g., Leu→Ser).

As a further option are alphavirus adjuvants comprising an alphavirus capsid protein (or a nucleic acid [e.g., DNA and/or RNA] encoding an alphavirus capsid protein) in which there is an attenuating mutation in the capsid protease that reduces, or even ablates, the autoprotease activity of the capsid and results, therefore, in non-viable virus. Capsid mutations that reduce or ablate the autoprotease activity of the alphavirus capsid are known in the art, see e.g., WO 96/37616 to Johnston et al. In particular embodiments, the alphavirus adjuvant comprises a VEE capsid protein in which the capsid protease is reduced or ablated, e.g., by introducing an amino acid substitution at VEE capsid position 152, 174, or 226. Alternatively, one or more of the homologous positions in other alphaviruses may be altered to reduce capsid protease activity.

If the alphavirus adjuvant comprises a Sindbis-group virus (e.g., Sindbis, TR339, S.A.AR86, Girdwood S A, Ockelbo) capsid protein, the attenuating mutation may be a mutation at capsid amino acid position 215 (e.g., a Ser→Ala) that reduces capsid autoprotease activity (see, Hahn et al., (1990) J. Virology 64:3069).

It is not necessary that the attenuating mutation(s) eliminate all pathology or adverse effects associated with administration of the viral adjuvant, as long as there is some improvement or benefit (e.g., increased safety and/or reduced morbidity and/or reduced mortality) as a result of the attenuating mutation.

In particular embodiments, the attenuating mutation is an attenuating mutation in one or more of the cleavage domains between the alphavirus nonstructural (nsp) genes, e.g., the nsP1/nsP2 cleavage region, the nsP2/nsP3 cleavage region, and/or the nsP3/nsP4 cleavage region as described in PCT Application No. PCT/US01/27644. An exemplary attenuating mutation is a mutation at S.A.AR86 nsP1 amino acid 538 (position P3), for example a substitution mutation at S.A.AR86 nsP1 amino acid 538, (e.g., a Thr→Ile substitution at S.A.AR86 nsP1 amino acid 538).

In particular embodiments, the attenuating mutation reduces (e.g., by at least 25%, 40%, 50%, 60%, 70%, 80%, 90%, 95% or more) the neurovirulence of the alphavirus adjuvant (e.g., as determined by intracerebral injection in weanling or adult mice).

Those skilled in the art may identify attenuating mutations other than those specifically disclosed herein using other methods known in the art, e.g., looking at neurovirulence in weanling or adult mice following intracerebral injection. Methods of identifying attenuating mutations in alphaviruses are described by Olmsted et al., (1984) Science 225:424 and Johnston and Smith, (1988) Virology 162:437).

Those skilled in the art will appreciate that in some embodiments, the viral adjuvant may have no pathological effects, but one or more attenuating mutations is included as a safety feature in the event that recombination gives rise to an infectious and otherwise pathogenic virus.

To identify other attenuating mutations other than those specifically disclosed herein, amino acid substitutions may be based on any characteristic known in the art, including the relative similarity or differences of the amino acid side-chain substituents, for example, their hydrophobicity, hydrophilicity, charge, size, and the like.

Amino acid substitutions other than those disclosed herein may be achieved by changing the codons of the genomic RNA sequence (or a DNA sequence), according to the following codon table:

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC ACU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

In identifying other attenuating mutations, the hydropathic index of amino acids may be considered. The importance of the hydropathic amino acid index in conferring interactive biologic function on a protein is generally understood in the art (see, Kyte and Doolittle, (1982) J. Mol. Biol. 157:105; incorporated herein by reference in its entirety). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte and Doolittle, Id.), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

Accordingly, the hydropathic index of the amino acid (or amino acid sequence) may be considered when identifying additional attenuating mutations according to the present invention.

It is also understood in the art that the substitution of amino acids can be made on the basis of hydrophilicity. U.S. Pat. No. 4,554,101 states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); threonine (+0.3); asparagine (+0.2); glutamine (+0.2); glycine (0); threonine (−0.4); proline (−0.5±I); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

Thus, the hydrophilicity of the amino acid (or amino acid sequence) may be considered when identifying additional attenuating mutations according to the present invention.

Mutations may be introduced into the alphavirus genome by any method known in the art. For example, mutations may be introduced into the alphavirus RNA by performing site-directed mutagenesis on the cDNA which encodes the RNA, in accordance with known procedures (see, Kunkel, Proc. Natl. Acad. Sci. USA 82, 488 (1985)). Alternatively, mutations may be introduced into the RNA by replacement of homologous restriction fragments in the cDNA which encodes for the RNA in accordance with known procedures.

### IV. Methods of Administration and Subjects.

The present invention can be practiced for prophylactic and/or therapeutic purposes, in accordance with known techniques. In addition, the invention can be practiced to produce antibodies for any purpose, such as diagnostic or research purposes, or for passive immunization by transfer to another subject.

To illustrate, the invention can be practiced to produce an immune response against a norovirus in a subject, optionally a protective immune response. With respect to a protective immune response, the present invention can be practiced prophylactically to prevent norovirus infection. In other embodiments, the methods of the invention are practiced to treat a subject infected by a norovirus.

Further, in representative embodiments, the methods of the invention can be practice to produce an immune response, optionally a protective immune response, against one or more norovirus genoclusters and/or strains that are not included in the composition or immunogenic formulation administered to the subject.

The present invention provides methods to concurrently induce an immune response (e.g., a protective immune response against two or more noroviruses (e.g., two, three, four, five, six, seven, eight, nine, ten or more). In some embodiments, an immune response (e.g., a protective immune response) is provided against one or more norovirus genoclusters and/or strains not included in the vaccine mix (e.g., cross-immunization or cross-protection), for example, GI.1 and/or GII.4.

In one embodiment, the methods of the invention induce humoral, mucosal and/or cellular immunity (optionally, protective immunity) against one or more (e.g., one, two, three, four, five, six, seven, eight, nine or ten, or more) of the norovirus genoclusters and/or strains included within the immunogenic composition, optionally all of the norovirus genoclusters and/or strains included in the composition.

In one embodiment, the composition induces humoral, mucosal and/or cellular immunity (optionally, protective immunity) against one or more norovirus genoclusters and/or strains (e.g., one, two, three, four, five, six, seven, eight, nine or ten or more) not included within the immunogenic composition.

In embodiments of the invention, the composition induces a strong carbohydrate blockade response (e.g., a neutralizing response) against at least one VLP receptor interactions (e.g., with one or more histo-blood group antigens such as H type 1 antigen and/or the H type 3 antigen, A antigen, B antigen and/or a Lewis antigen including without limitation Le^{a}, Le^{b}, Le^{x}). In particular embodiments, at least one VLP receptor interaction is reduced by at least about 25%, 35%, 50%, 60%, 70%, 80%, 90%, 95%, 97%, 98% or more.

Immunogenic formulations for use in the inventive methods are described below. Boosting dosages can further be administered over a time course of days, weeks, months or years. In chronic infection, initial high doses followed by boosting doses may be advantageous.

The present invention can be practiced for both medical and veterinary purposes. Subjects to be treated by the methods of the invention can include avian and/or mammalian subjects.

Suitable subjects include both males and females and subjects of all ages including infant, juvenile, adolescent, adult and geriatric subjects. Subjects may be treated for any purpose, such as for eliciting a protective immune response; or for eliciting the production of antibodies in that subject, which antibodies can be collected and used for other purposes such as research or diagnostic purposes or for administering to other subjects to produce passive immunity therein, etc.

In embodiments of the invention, the subject is a child less than about 5 years of age. In other representative embodiments, the subject is a child less than about 2 years of age (e.g., a toddler or an infant).

In embodiments of the invention, the subject is an immunocompromised subject (e.g., a subject with HIV/AIDS, a subject with cancer, a subject undergoing chemotherapy, radiation therapy, a subject following bone marrow transplant and/or a subject following organ transplant).

In embodiments, the subject is a geriatric subject, optionally a geriatric subject living in an institutional setting (e.g., a hospital or nursing home).

In embodiments, the subject is a member of the military, e.g., a member of the military living on base or on a ship.

Accordingly, as one aspect, the invention provides a method of producing an immune response against a norovirus (e.g., two or more noroviruses) in a subject, the method comprising administering an immunogenically effective amount of a composition or immunogenic formulation of the invention to the subject.

The invention further provides a method of protecting a subject from norovirus infection (e.g., from infection with two or more noroviruses), the method comprising administering a composition or immunogenic formulation of the invention to the subject in an amount effective to protect the subject from norovirus infection.

Also provided is a method of preventing norovirus infection (e.g., infection with two or more noroviruses), the method comprising administering a composition or immunogenic formulation of the invention to the subject in an amount effective to prevent norovirus infection in the subject.

Also contemplated is a method of treating norovirus infection (e.g., infection with two or more noroviruses), the method comprising administering a composition or immunogenic formulation of the invention to the subject in an amount effective to treat norovirus infection in the subject.

The invention also provides a method of producing an immune response against a norovirus (e.g., two or more noroviruses) in a subject, the method comprising administering to the subject:

(a) an immunogenically effective amount of a composition or immunogenic formulation of the invention; and

(b) an adjuvant.

The invention also provides a method of protecting a subject from norovirus infection (e.g., from infection with two or more noroviruses), the method comprising administering to the subject:

(a) a composition or immunogenic formulation of the invention in an amount effective to protect the subject from norovirus infection; and

(b) an adjuvant.

Further provided is a method of preventing norovirus infection in a subject (e.g., infection with two or more noroviruses), the method comprising administering to the subject:

(a) a composition or immunogenic formulation of the invention in an amount effective to prevent norovirus infection in the subject; and

(b) an adjuvant.

The present invention also encompasses a method of treating norovirus infection in a subject (e.g., infection with two or more noroviruses), the method comprising administering to the subject:

(a) a composition or immunogenic formulation of the invention in an amount effective to treat norovirus infection in the subject; and

(b) an adjuvant.

The adjuvant can be administered to the subject concurrently (in the same or separate compositions) or serially in any order.

Those skilled in the art will appreciate that the one or more booster dosages can be administered.

Administration can be by any route known in the art. As non-limiting examples, the route of administration can be by inhalation (e.g., oral and/or nasal inhalation), oral, buccal (e.g., sublingual), rectal, vaginal, topical (including administration to the airways), intraocular, transdermal, by parenteral (e.g., intramuscular [e.g., administration to skeletal muscle], intravenous, intra-arterial, intraperitoneal and the like), subcutaneous, intradermal, intrapleural, intracerebral, and/or intrathecal routes.

In particular embodiments, administration is to a mucosal surface, e.g., by intranasal, inhalation, intra-tracheal, oral, buccal (e.g., sublingual), intra-ocular, rectal or vaginal administration, and the like. In general, mucosal administration refers to delivery to a mucosal surface such as a surface of the respiratory tract, gastrointestinal tract, urinary tract, reproductive tract, etc.

Methods of administration to the respiratory tract include but are not limited to transmucosal, intranasal, inhalation, bronchoscopic administration, or intratracheal administration or administration to the lungs.

The norovirus VLPs and/or viral adjuvants can be delivered per se or by delivering a nucleic acid that encodes the norovirus VLPs and/or viral adjuvant.

Immunomodulatory compounds, such as immunomodulatory chemokines and cytokines (preferably, CTL inductive cytokines) can be administered concurrently to a subject.

Cytokines may be administered by any method known in the art. Exogenous cytokines may be administered to the subject, or alternatively, a nucleic acid encoding a cytokine may be delivered to the subject using a suitable vector, and the cytokine produced in vivo. In particular embodiments, a viral adjuvant expresses the cytokine.

### V. Pharmaceutical Formulations.

The invention further provides pharmaceutical formulations (e.g., immunogenic formulations) comprising a composition of the invention comprising two or more norovirus VLPs in a pharmaceutically acceptable carrier. In particular embodiments, the pharmaceutical composition is formulated for mucosal, intradermal, intramuscular or subcutaneous delivery. By “pharmaceutically acceptable” it is meant a material that is not toxic or otherwise undesirable. Optionally, the pharmaceutical formulation further comprises one or more adjuvants.

In representative embodiments, the composition is present in the pharmaceutical formulation in an “immunogenically effective” amount. An “immunogenically effective amount” is an amount that is sufficient to evoke an active immune response (i.e., cellular and/or humoral) in the subject to which the pharmaceutical formulation is administered, optionally a protective immune response. The degree of protection conferred need not be complete or permanent, as long as the benefits of administering the pharmaceutical formulation outweigh any disadvantages thereof.

In embodiments of the invention, the dosage of each VLP in the immunogenic formulations of the invention is greater than or equal to about 0.01, 0.1, 0.5, 0.75, 1, 2, 3, 4 or 5 μg and/or less than or equal to about 4, 5, 6, 8, 10, 12, 15, 20, 25, 30, 40, 50, 60, 70, 80, 90 or 100 μg or more (encompassing any combination as long as the lower limit is less than the upper limit).

In representative embodiments, the formulation comprises a viral adjuvant. With respect to viral adjuvants, in particular embodiments, the dosage of the viral adjuvant is greater than or equal to about 10⁻², 10⁻¹, 10, 10², 10³, 10⁴, 10⁵ or 10⁶ virus particles, virus-like particles, or infectious units and/or less than or equal to about 10², 10³, 10⁴, 10⁵, 10⁶, 10⁷, 10⁸, 10⁹, 10¹⁰, 10¹¹, 10¹², 10¹³ or even 10¹⁴ or more virus particles, virus-like particles, or infectious units (encompassing any combination as long as the lower limit is less than the upper limit). Those skilled in the art will appreciate that some methods of titering viruses have relatively low sensitivity giving rise to apparent titers of less than one virus particle. Viral titers can be assessed by any method known in the art, including cytotoxicity in cultured cells (e.g., alphavirus titers can be assessed by cytotoxicity in BHK cells). In other representative embodiments, a dosage of about 10⁻¹ to 10⁷, 10 to 10⁶ or about 10² to 10⁴ virus particles, virus-like particles, or infectious units are administered.

Further, in some embodiments, an adjuvant is present in an “adjuvant effective amount.”

The pharmaceutical formulations of the invention can optionally comprise other medicinal agents, pharmaceutical agents, stabilizing agents, buffers, carriers, diluents, salts, tonicity adjusting agents, wetting agents, and the like, for example, sodium acetate, sodium lactate, sodium chloride, potassium chloride, calcium chloride, sorbitan monolaurate, triethanolamine oleate, etc.

For injection, the carrier will typically be a liquid. For other methods of administration, the carrier may be either solid or liquid. For inhalation administration, the carrier will be respirable, and is typically in a solid or liquid particulate form.

The compositions of the invention can be formulated for administration in a pharmaceutical carrier in accordance with known techniques. See, e.g., Remington, The Science And Practice of Pharmacy (9^{th} Ed. 1995). In the manufacture of a pharmaceutical composition according to the invention, the VLPs are typically admixed with, inter alia, an acceptable carrier. The carrier can be a solid or a liquid, or both, and is optionally formulated with the compound as a unit-dose formulation, for example, a tablet. A variety of pharmaceutically acceptable aqueous carriers can be used, e.g., water, buffered water, 0.9% saline, 0.3% glycine, hyaluronic acid, pyrogen-free water, pyrogen-free phosphate-buffered saline solution, bacteriostatic water, or Cremophor EL[R] (BASF, Parsippany, N.J.), and the like. These compositions can be sterilized by conventional techniques. The formulations of the invention can be prepared by any of the well-known techniques of pharmacy.

The pharmaceutical formulations can be packaged for use as is, or lyophilized, the lyophilized preparation generally being combined with a sterile aqueous solution prior to administration. The compositions can further be packaged in unit/dose or multi-dose containers, for example, in sealed ampoules and vials.

The pharmaceutical formulations can be formulated for administration by any method known in the art according to conventional techniques of pharmacy. For example, the compositions can be formulated to be administered intranasally, by inhalation (e.g., oral inhalation), orally, buccally (e.g., sublingually), rectally, vaginally, topically, intrathecally, intraocularly, transdermally, by parenteral administration (e.g., intramuscular [e.g., skeletal muscle], intravenous, subcutaneous, intradermal, intrapleural, intracerebral and intra-arterial, intrathecal), or topically (e.g., to both skin and mucosal surfaces, including airway surfaces).

For intranasal or inhalation administration, the pharmaceutical formulation can be formulated as an aerosol (this term including both liquid and dry powder aerosols). For example, the pharmaceutical formulation can be provided in a finely divided form along with a surfactant and propellant. Typical percentages of the composition are 0.01-20% by weight, preferably 1-10%. The surfactant is generally nontoxic and soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1-20% by weight of the composition, preferably 0.25-5%. The balance of the composition is ordinarily propellant. A carrier can also be included, if desired, as with lecithin for intranasal delivery. Aerosols of liquid particles can be produced by any suitable means, such as with a pressure-driven aerosol nebulizer or an ultrasonic nebulizer, as is known to those of skill in the art. See, e.g., U.S. Pat. No. 4,501,729. Aerosols of solid particles can likewise be produced with any solid particulate medicament aerosol generator, by techniques known in the pharmaceutical art. Intranasal administration can also be by droplet administration to a nasal surface.

Injectable formulations can be prepared in conventional forms, either as liquid solutions or suspensions, solid forms suitable for solution or suspension in liquid prior to injection, or as emulsions. Alternatively, one can administer the pharmaceutical formulations in a local rather than systemic manner, for example, in a depot or sustained-release formulation.

Extemporaneous injection solutions and suspensions can be prepared from sterile powders, granules and tablets of the kind previously described. For example, an injectable, stable, sterile formulation of the invention in a unit dosage form in a sealed container can be provided. The formulation can be provided in the form of a lyophilizate, which can be reconstituted with a suitable pharmaceutically acceptable carrier to form a liquid composition suitable for injection into a subject. The unit dosage form can be from about 1 jig to about 10 grams of the formulation. When the formulation is substantially water-insoluble, a sufficient amount of emulsifying agent, which is pharmaceutically acceptable, can be included in sufficient quantity to emulsify the formulation in an aqueous carrier. One such useful emulsifying agent is phosphatidyl choline.

Pharmaceutical formulations suitable for oral administration can be presented in discrete units, such as capsules, cachets, lozenges, or tables, as a powder or granules; as a solution or a suspension in an aqueous or non-aqueous liquid; or as an oil-in-water or water-in-oil emulsion. Oral delivery can be performed by complexing a compound(s) of the present invention to a carrier capable of withstanding degradation by digestive enzymes in the gut of an animal. Examples of such carriers include plastic capsules or tablets, as known in the art. Such formulations are prepared by any suitable method of pharmacy, which includes the step of bringing into association the protein(s) and a suitable carrier (which may contain one or more accessory ingredients as noted above). In general, the pharmaceutical formulations are prepared by uniformly and intimately admixing the compound(s) with a liquid or finely divided solid carrier, or both, and then, if necessary, shaping the resulting mixture. For example, a tablet can be prepared by compressing or molding a powder or granules, optionally with one or more accessory ingredients. Compressed tablets are prepared by compressing, in a suitable machine, the formulation in a free-flowing form, such as a powder or granules optionally mixed with a binder, lubricant, inert diluent, and/or surface active/dispersing agent(s). Molded tablets are made by molding, in a suitable machine, the powdered protein moistened with an inert liquid binder.

Pharmaceutical formulations suitable for buccal (sub-lingual) administration include lozenges comprising the compound(s) in a flavored base, usually sucrose and acacia or tragacanth; and pastilles in an inert base such as gelatin and glycerin or sucrose and acacia.

Pharmaceutical formulations suitable for parenteral administration can comprise sterile aqueous and non-aqueous injection solutions, which preparations are preferably isotonic with the blood of the intended recipient. These preparations can contain anti-oxidants, buffers, bacteriostats and solutes, which render the composition isotonic with the blood of the intended recipient. Aqueous and non-aqueous sterile suspensions, solutions and emulsions can include suspending agents and thickening agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical formulations suitable for rectal administration are optionally presented as unit dose suppositories. These can be prepared by admixing the active agent with one or more conventional solid carriers, such as for example, cocoa butter and then shaping the resulting mixture.

Pharmaceutical formulations suitable for topical application to the skin preferably take the form of an ointment, cream, lotion, paste, gel, spray, aerosol, or oil. Carriers that can be used include, but are not limited to, petroleum jelly, lanoline, polyethylene glycols, alcohols, transdermal enhancers, and combinations of two or more thereof. In some embodiments, for example, topical delivery can be performed by mixing a pharmaceutical formulation of the present invention with a lipophilic reagent (e.g., DMSO) that is capable of passing into the skin.

Pharmaceutical formulations suitable for transdermal administration can be in the form of discrete patches adapted to remain in intimate contact with the epidermis of the subject for a prolonged period of time. Formulations suitable for transdermal administration can also be delivered by iontophoresis (see, for example, Pharmaceutical Research 3:318 (1986)) and typically take the form of a buffered aqueous solution of the compound(s). Suitable formulations can comprise citrate or bis\tris buffer (pH 6) or ethanol/water and can contain from 0.1 to 0.2M active ingredient.

Further, the composition can be formulated as a liposomal formulation. The lipid layer employed can be of any conventional composition and can either contain cholesterol or can be cholesterol-free. The liposomes that are produced can be reduced in size, for example, through the use of standard sonication and homogenization techniques.

The liposomal formulations can be lyophilized to produce a lyophilizate which can be reconstituted with a pharmaceutically acceptable carrier, such as water, to regenerate a liposomal suspension.

The immunogenic formulations of the invention can optionally be sterile, and can further be provided in a closed pathogen-impermeable container.

The present invention is more particularly described in the following examples that are intended as illustrative only since numerous modifications and variations therein will be apparent to those skilled in the art.

### EXAMPLE 1

### Materials & Methods

Virus Like Particles (VLPs) and Venezuelan Equine Encephalitis Virus Replicon Particles (VRPs). VRPs expressing norovirus open reading frame 2 were cloned and produced as described in reference 6. Experimental use of VLPs derived from the Southampton (SoV), Chiba, Desert Shield (DSV), Toronto (TV), and M7 virus strains and produced using the VRP system has not been described previously. Null VRPs were kindly provided by the Carolina Vaccine Institute (UNC). Norovirus VLPs were produced and purified as described in reference 31 and visualized by electron microscopy to ensure appropriate particle size and structure. VLPs used in vaccination experiments were further concentrated by centrifugation at 3,000×g in Centricon tubes (Millipore) overnight at 4° C.

Vaccination. Six-week-old BALB/c mice (Charles River) were vaccinated by footpad inoculation in two independent experiments with monovalent or multivalent VLP vaccines containing 2 kg of each VLP alone or in conjunction with 10⁵ null VRP or 1 μg oligodeoxynucleotide 1826 CpG DNA (5′-TCCATGACGTTCCTGACGTT-3′ (SEQ ID NO:2); Invivogen) (n=4 per vaccination group). Mice used in. VLP titration experiments received VLP doses of 0.02 μg, 0.2 μg, 2 μg, or 10 μg Norwalk virus (NV) VLP coadministered with null VRP (n=4 per group). Other monovalent vaccination groups received NV (genogroup I.1 [GI.1]), Lordsdale-like virus (LV; GII.4), or MNV-1 (GV) VLPs. Multivalent groups received GI-specific VLPs representing SoV (GI.2), DSV (GI.3), and Chiba (GI.4) strains with or without NV VLPs; GII-specific VLPs representing Hawaii (GII.1), TV (GII.3), and M7 (GII.13) strains with or without LV VLPs; or complete VLP cocktails containing all GI and GII VLPs with or without NV and LV VLPs or all GI and GII VLPs with or without MNV VLPs (GV) (Table 1). Mice were vaccinated and boosted at days 0 and 28. Donor mice for adoptive transfers (n=8) were vaccinated a third time on day 52.

MNV infection. MNV-1 strain CW.3 was kindly provided by H. W. Virgin (Washington University School of Medicine). To generate virus stocks, murine macrophage-like raw 264.7 cells (UNC Tissue Culture Facility) cultured in complete Dulbecco's modified Eagle medium (Gibco) were infected with MNV at a multiplicity of infection of 0.1 and incubated for 36 h. Supernatant was then collected, clarified by centrifugation at 13,000×g for 15 min (Beckman), and ultracentrifuged for 3 h at 100,000×g over a 5% sucrose cushion to pellet purified virus. Pellets were resuspended in phosphate-buffered saline (PBS), aliquoted, and stored at −80° C. until use. Titers of virus stocks were determined by plaque assay as previously described (56). Mice used in MNV challenge experiments were infected with 3×10⁷ PFU MNV-1 strain CW.3 in 30 μl total volume orally on day 42 postvaccination.

Serum samples, fecal extracts, and tissue samples. Animals were euthanized and distal ileum, spleen, mesenteric lymph node (MLN), and serum samples were harvested from mice used in MNV challenge experiments on day 45 and stored at −80° C. Tissue samples were resuspended in 1 ml complete Dulbecco's modified Eagle medium and disrupted with silica/zirconia beads (Biospec Products) using the MagnaLyser homogenizer (Roche) at 6,000 rpm for 30 s. Serum and fecal samples from all other mice were collected on day 42. Ten fecal pellets per mouse were resuspended in 1 ml PBS containing 10% goat serum and 0.01% Kathon fecal inactivator (Supelco) and homogenized by vortexing for 20 min. Solid material was then removed by centrifugation for 20 min, and fecal extracts were stored at −20° C.

ELISA and HBGA binding blockade assays. Enzyme-linked immunosorbent assays (ELISAs) for serum immunoglobulin G (IgG) antibody cross-reactivity to norovirus VLPs and binding assays for serum antibody blockade of HBGA binding were performed as previously described (31). IgG subtype ELISAs were performed as described using purified IgG1 (Sigma) or IgG2a (Sigma) as the standard control and anti-IgG1-alkaline phosphatase (Southern Biotech) and anti-IgG2a-alkaline phosphatase (Southern Biotech) as secondary antibodies. The lower limit of detection for all serum ELISAs ranged from 0.1 to 1.9 μg/ml and was assay dependent. To quantitate specific antibody in fecal extracts, 96-well high-binding plates (Costar) were coated with 2 μg VLP or serially diluted mouse IgG or IgA standard for 4 h at room temperature and blocked overnight in blocking buffer (Sigma) at 4° C. Fecal extracts diluted 1:2 in blocking buffer were twofold serially diluted and incubated in wells containing VLP for 2 h at room temperature. Wells were then incubated with antimouse IgG-horseradish peroxide or IgA-horseradish peroxide (Southern Biotech) for 2 h and developed with orthophenylene-diamine tablets (Sigma) dissolved in 1:1 0.1 M sodium citrate and 0.1 M citric acid and 0.02% hydrogen peroxide for 30 min in the dark. Reactions were stopped with 0.1 M sodium fluoride, and the optical density at 450 nm was read (Bio-Rad model 680). The limit of detection for fecal IgG and IgA ELISAs was 0.2 ng/ml. All data are representative of the results for two independent vaccination experiments.

Passive and adoptive transfers. Eight wild-type mice were immunized as described above, and unimmunized controls were treated in parallel. Serum samples and spleens from immune and control groups were harvested on day 56. Spleens from respective immunization groups were pooled, and single-cell splenocyte suspensions were obtained by manual disruption through a 100-μm cell strainer. Splenocyte suspensions were resuspended in MACS buffer (PBS [pH 7.2], 0.5% bovine serum albumin, 2 mM EDTA) and divided in half, and CD4⁺ or CD8⁺ cells were purified, respectively, by magnetic bead sorting using the QuadroMACS purification system (Miltenyi) per manufacturer's protocol. For adoptive transfers, 5×10⁶CD4⁺ or CD8⁺ cells from immune or nonimmune mice were administered in a total volume of 500 μl intraperitoneally (i.p.) to wild-type BALB/c mice or SCID C.B.17 mice (Jackson Laboratories) (n=6 per recipient group). For passive transfer of sera, immune or nonimmune serum samples were equivalently pooled, diluted 1:2 in PBS, and administered i.p. to recipient mice at 200 μl per mouse. Recipient mice were challenged with 3×10⁷ PFU MNV CW.3 24 hours posttransfer, and tissues were harvested 3 days postinfection. Tissue samples were processed as described above.

FACS. Whole and purified splenocyte suspensions from adoptive transfer groups were set aside for fluorescence-activated cell sorter (FACS) analysis. A total of 5×10⁵ cells per tube were blocked with anti-FcΛIII (1:500; eBioscience) in 100 μl FACS buffer (Hank's balanced salt solution plus 2% fetal bovine serum) for 20 min on ice. Cells were then pelleted, resuspended in 100 μl FACS buffer, and stained with anti-B220 conjugated to fluorescein isothiocyanate (1:400), allophycocyanin (1:400), or biotin (1:800) as single color controls for staining or cocktails containing anti-CD3-fluorescein isothiocyanate (1:200), anti-CD4-biotin (1:1,000), and anti-CD8-allophycocyanin (1:800). Cells were incubated for 45 min on ice, pelleted, and resuspended in 100 μl FACS buffer with avidin-PerCP (1:400) for 45 min on ice. Samples were then washed and resuspended in 500 μl PBS. All antibodies were obtained from eBioscience (San Diego, Calif.). FACS analysis was performed by the UNC Flow Cytometry Core Facility.

Statistics. All statistics comparing two groups were performed using the two-tailed t test; all statistics comparing multiple groups were performed using one-way analysis of variance and Tukey's posttest in GraphPad software.

### EXAMPLE 2

### Results

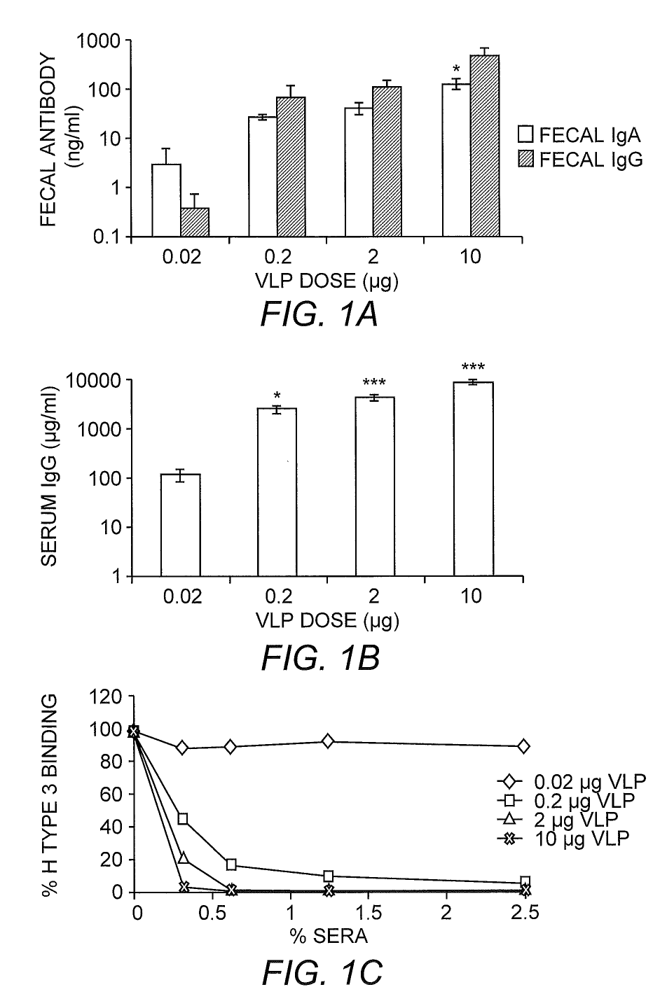

Null VRP adjuvants induce robust systemic and mucosal antibody responses in monovalent VLP vaccines. To determine effective VLP concentrations for subsequent vaccinations, mice were immunized twice with a VLP titration series consisting of 10 μg, 2 μg, 0.2 μg, or 0.02 μg NV VLPs coadministered with 10⁵ IU null VRPs. Fecal IgA, fecal IgG, serum IgG, and serum blockade of receptor binding were evaluated 3 weeks postboost (FIG. 1). Measurable IgA and IgG were detected in fecal extracts of all mice receiving 0.2 to 10 μg VLPs in the presence of VRP adjuvants (FIG. 1A). Antibody titers were increased following vaccination with increasing amounts of VLP, and fecal IgG titers were consistently higher than fecal IgA titers, in line with previous results obtained with VEE adjuvants (49, 50). Serum antibody responses were also significantly increased following vaccination with all VLP concentrations at >0.02 μg compared to vaccination with 0.02 μg VLP (P<0.05) (FIG. 1 B) and blocked H type 3 receptor binding increasingly effectively with increased VLP concentration (FIG. 1C). From these data, we concluded that a dose of 2 μg of VLPs elicited a robust humoral immune response in rodents, and as such, all subsequent multivalent vaccine experiments were performed with this dose. Of note, multivalent vaccines cannot accommodate all VLPs at higher concentrations (i.e., 10 μg per VLP) due to footpad volume restrictions.

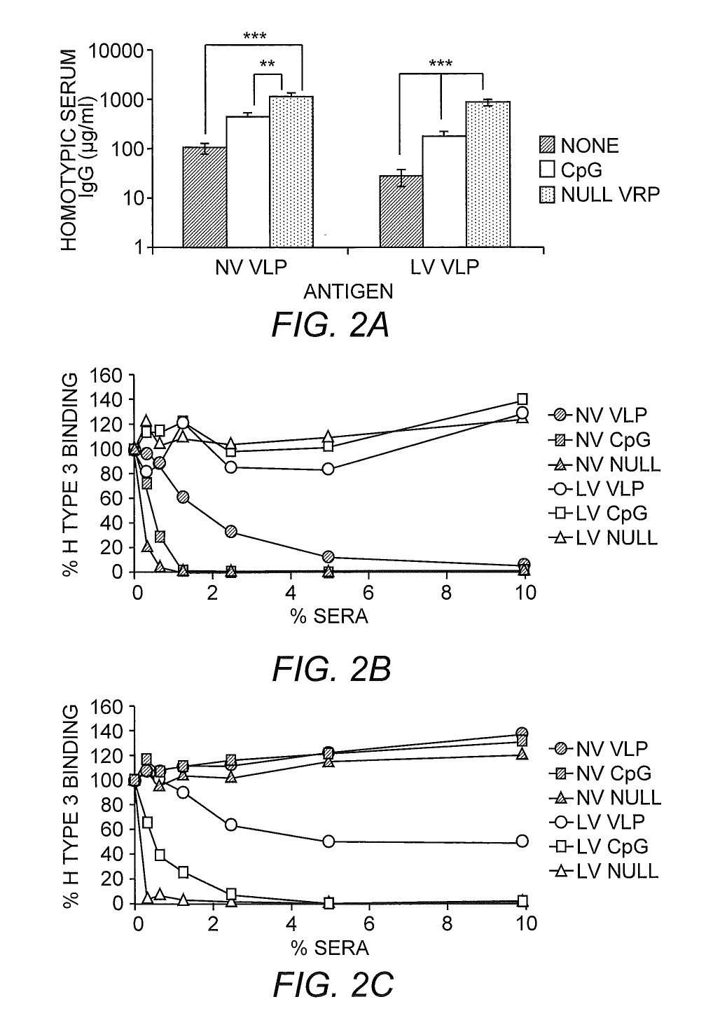

To compare the effect of null VRP adjuvant activity to that of an FDA-approved adjuvant for human vaccination, we immunized mice with 2 μg NV VLPs or LV VLPs alone or in conjunction with either 10⁵ IU null VRPs or 1 μg CpG DNA (25). Serum antibody responses to NV or LV VLPs were significantly increased following codelivery with null VRPs compared to with low-dose CpG adjuvants (P<0.01 and P<0.001, respectively), and both adjuvant groups induced significantly higher responses than did VLP alone (P<0.001) (FIG. 2A). Sera from groups vaccinated with NV VLPs but not LV VLPs blocked NV VLP binding to H type 3, and adjuvanted groups blocked binding with serum concentrations lower than those of groups receiving VLPs alone (FIG. 2B). Parallel results were obtained for blockade of LV VLP binding to H type 3 following LV VLP vaccination, respectively (FIG. 2C). Percentages of sera necessary for blockade of 50% (BT50) and 90% (BT90) H type 3 binding are shown in Table 2. BT50 and BT90 values were significantly lower in adjuvanted sera than in nonadjuvanted sera (P<0.05).

Multivalent vaccines induce enhanced cross-reactive and receptor-blocking antibody responses. To determine the effect of multivalent VLP vaccination with null VRP or CpG adjuvants on homotypic and heterotypic antibody responses and receptor blockade, we vaccinated mice with pools of VLPs (2 μg each VLP) alone or coadministered with null VRP or CpG adjuvants. Mice received multivalent immunizations consisting of GI VLPs, GII VLPs, or both GI and GII VLPs. GI VLPs are derived from the NV (GI.1), SoV (GI.2), DSV (GI.3), and Chiba (GI.4) strains, and the GII VLPs are derived from the LV (GII.4), Hawaii (GII.1), TV (GII.3), and M7 (GII.13) strains. VLP vaccine formulations and acronyms are summarized in Table 1. NV VLPs were excluded from GI-specific (GI−) and complete GI/GII (GI−/GII−) multivalent vaccine formulations to allow comparison of their heterotypic antibody blockade of receptor binding to NV VLPs with that elicited by vaccines containing the NV antigen. LV VLPs were likewise excluded from GII-specific (GII−) and complete (GI−/GII−) vaccine formulations. Serum IgG responses following vaccination with the complete cocktail of GI/GII VLPs (GI+/GII+) coadministered with null VRP adjuvants resulted in robust antibody responses to NV and LV VLPs, respectively, that were significantly higher than those in groups lacking adjuvant (P<0.001) (FIG. 3A). Furthermore, antisera following GI−/GII− vaccination still mounted strong cross-reactive IgG responses to NV and LV VLPs. GI−/GII−VLP pools coadministered with null VRPs induced significantly stronger heterotypic responses to NV and LV VLPs than did GI−/GII−VLP vaccination without adjuvant (P<0.05). However, GI−/GII− heterotypic antiserum reactivity to NV and LV VLPs was significantly lower than that of homotypic GI+/GII+ antisera (P<0.05). Evaluation of antiserum blockade of H type 3 binding to VLPs revealed that GI+/GII+ antisera completely blocked H type 3 binding to both NV and LV VLPs, with increased blockade in groups receiving adjuvant (FIG. 3B to C). Significantly less serum was required to attain BT90 values following GI+/GII+ vaccination with adjuvant than without adjuvant (Table 2). Furthermore, GI−/GII− antisera induced by the GI−/GII− vaccine plus null VRPs contained cross-reactive antibodies that partially ablated H type 3 binding to both NV and LV VLPs. BT50 serum concentrations were significantly higher following GI−/GII− null VRP vaccination than those following GI+/GII+ null VRP vaccination in NV VLP H type 3 blockade (P<0.05); however, they were not significantly different in LV VLP H type 3 blockade. Also, BT50 concentrations were significantly lower following GI−/GII− null VRP vaccination than those following GI−/GII−VLP vaccination without adjuvant (P<0.001 in NV blockade and P<0.05 in LV blockade). These data suggest that multivalent vaccines coadministered with null VRP adjuvants efficiently induce cross-reactive and receptor-blocking IgG responses to heterologous strains that cannot be attained following monovalent vaccination.

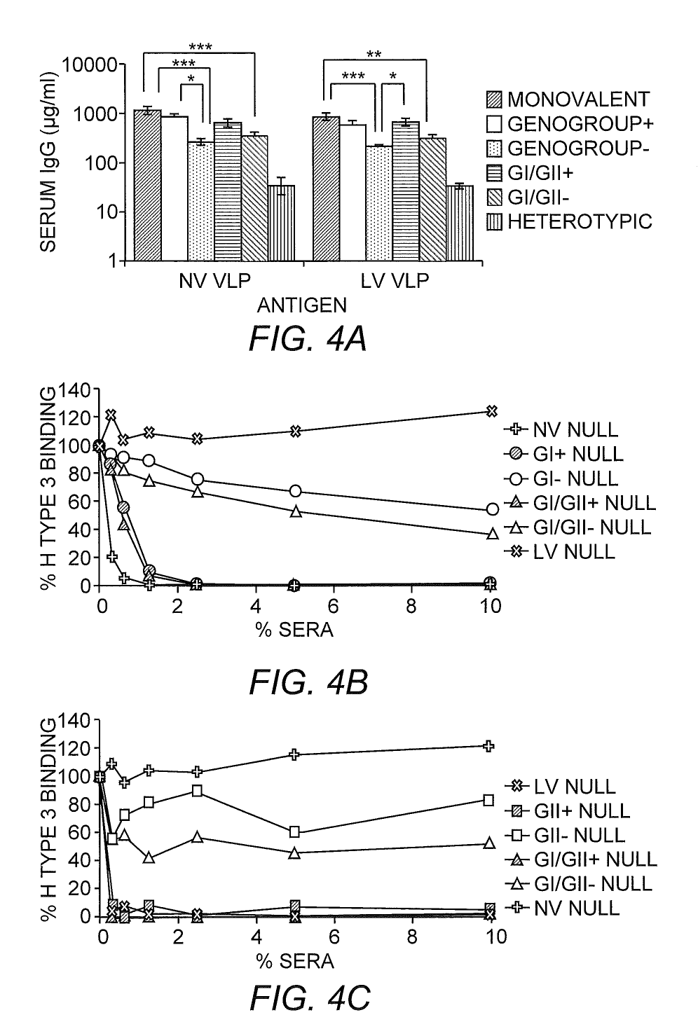

We performed an additional study in which mice were vaccinated with genogroup-specific VLP pools in conjunction with null VRP adjuvants. Groups of mice received immunizations of all four GI VLPs (GI+), all four GII VLPs (GII+), or three genogroup-specific VLPs lacking NV or LV VLPs (GI− and GII−, respectively) (Table 1). A comparison of serum IgG responses of genogroup-specific vaccinations to monovalent or multigenogroup VLP vaccines is shown in FIG. 4A. Cross-reactive responses of monovalent NV antisera to LV VLP and vice versa are shown as controls. All monovalent or multivalent vaccines containing NV or LV VLPs, respectively, induced highly reactive IgG responses to NV or LV VLPs that were not significantly different from one another. Genogroup-specific or multigenogroup VLP pools lacking NV and/or LV, respectively, mounted cross-reactive responses that were not significantly different from one another and were significantly lower than homotypic monovalent responses (P<0.01) only, but not homotypic multivalent responses. Blockade profiles from each genogroup-specific vaccination group uphold the findings discussed above whereby multivalent genogroup-specific vaccines lacking target antigens mount intermediate blockade responses (FIGS. 4B and C) with BT50 values significantly higher than homotypic values (P<0.05) but significantly lower than heterotypic monovalent values (P<0.01) (Table 2). Furthermore, increasing the number of VLPs in the vaccine composition did not significantly change homotypic antibody titers or blockade of receptor binding. Increasing genogroup-specific VLP vaccines to include VLPs from both genogroups appeared to moderately increase cross-reactive responses to both NV and LV VLPs, respectively. Increasing the amount of null VRPs administered from 10⁵ IU to 10⁶ IU per vaccine did not enhance cross-reactive receptor blockade responses (data not shown).

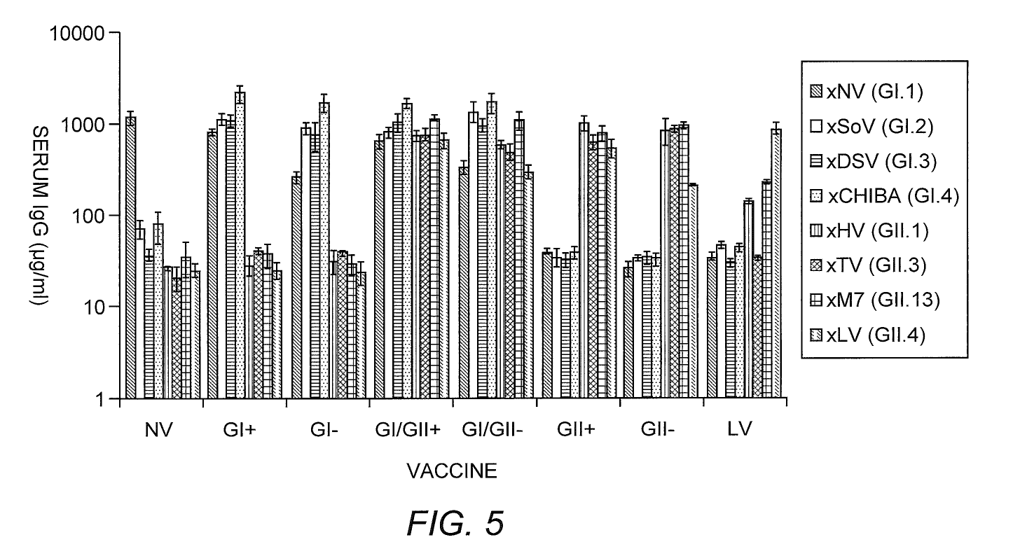

Complete profiles of cross-reactivity of all null VRP antiserum groups to the entire panel of VLPs are shown in FIG. 5. Obvious trends that emerge are significantly low cross-reactivity to additional VLPs following monovalent vaccination with NV or LV (P<0.001), although slightly increased cross-reactivity exists to VLPs within a genogroup; low cross-reactivity to strains in opposite genogroups following GI and GII vaccination (P<0.05); enhanced cross-reactivity to heterologous NV or LV strains within a genogroup following GI− and GII− vaccination, respectively; and cumulative cross-reactivity to heterologous NV and LV strains following complete VLP vaccination. These results suggest cross-reactivity induced by multivalent vaccination is likely genogroup-specific; therefore, vaccines must contain both GI and GII strains to induce a cumulative cross-reactivity to the majority of human norovirus strains.

Because noroviruses are enteric pathogens, a likely site of neutralization is the gastrointestinal tract. We, therefore, analyzed NV-specific IgG and IgA content in fecal extracts following monovalent or multivalent VLP vaccination coadministered with no adjuvant, CpG or null VRP (Table 3). NV-reactive IgG and IgA content, as well as total IgG and IgA content, was determined, and percentages of NV-specific subtype antibody were calculated. Significantly more total IgA than IgG was present in all fecal extracts tested (P<0.001); however, the percentage of IgG specific for NV VLPs was significntly higher than that of specific IgA in all samples (P<0.001). Vaccines coadministered with null VRP adjuvant induced significantly more total IgG, but not total IgA, than did covaccination with CpG (P<0.05) or VLP alone (P<0.01). A similar trend was seen by increasing the total number of VLPs administered in the vaccine composition, although values were not significant. Monovalent NV vaccination with null VRP induced significantly higher NV-specific IgA responses than did GI+/GII+ vaccination (P<0.05). Conversely, GI+/GII+ vaccination induced higher NV-specific IgG responses than did monovalent vaccination, although values were not significant. Multivalent GI−/GII− null VRP vaccination induced significantly lower NV-specific IgG (P<0.05), but not NV-specific IgA, than did GI+/GII+ vaccination. Percentages of NV-specific IgG were equivalent in NV and GI+/GII+ groups receiving either CpG or null VRP adjuvant; furthermore, the presence of adjuvant resulted in a substantial increase in total measurable IgG. Percentages of NV-specific IgA, however, were miniscule. LV-specific responses following monovalent and multivalent LV VLP vaccination were lower and more variable (data not shown). These data suggest that multivalent null VRP vaccination induces a predominantly IgG subtype response in the intestinal tract.

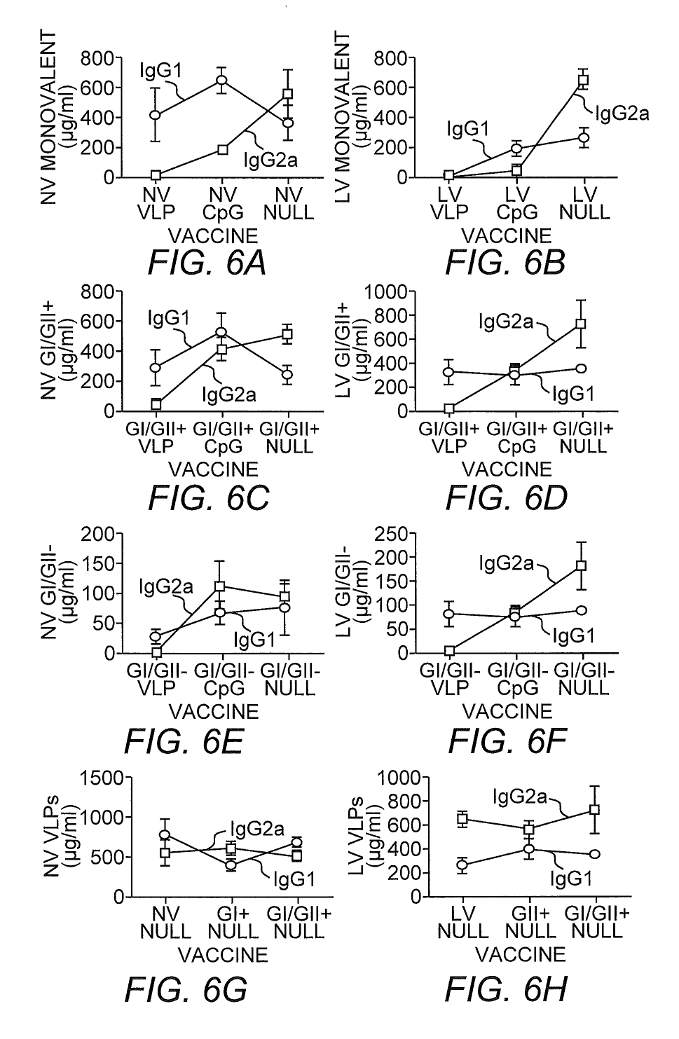

Null VRP vaccines induce stimulation of TH1-like IgG subclass responses. Previous studies have reported the activation of CD4⁺ T helper 1 (TH1) cells and the production of IFN-γ following norovirus infection (28). Because TH1 responses correlate with serum IgG2a subclass responses in mice, we used this alternative evaluation to determine induction of TH1 cell responses by multivalent VLP vaccination. Serum samples from mice vaccinated with monovalent or multivalent VLP vaccines alone or in conjunction with CpG or null VRP adjuvants were analyzed for IgG1 and IgG2a subclass specificity to NV and/or LV VLPs (FIG. 6). Monovalent and multivalent vaccination with NV and/or LV VLPs induced IgG2a titers that were slightly increased when coadministered with CpG and significantly increased when coadministered with null VRPs compared to when coadministered with VLPs alone (P<0.05). Heterotypic IgG2a responses to NV and LV VLPs following GI−/GII− vaccination were lower than homotypic responses, and titers were not different in CpG and null VRP recipient groups. IgG1 titers were not significantly different in VLP versus adjuvant groups but maintained uniform levels of reactivity to NV and LV VLPs that were significantly lower than IgG2a titers in null VRP recipient groups (P<0.05), although a spike in NV-specific IgG1 levels appeared to occur following monovalent and multivalent VLP vaccination with CpG. Increasing the number of VLPs in NV or LV null VRP vaccines from one to four to eight VLPs did not change IgG1 or IgG2a responses specific for NV or LV VLPs, respectively. Together, these data suggest that null VRP vaccines induce IgG2a responses specific for NV and/or LV antigens, which may correlate with a TH1-type response. Furthermore, CpG and null VRP adjuvants induced cross-reactive IgG2a to NV and LV VLPs in the GI−/GII− vaccine group, implying that TH1 cross-reactivity to additional strains may also occur.

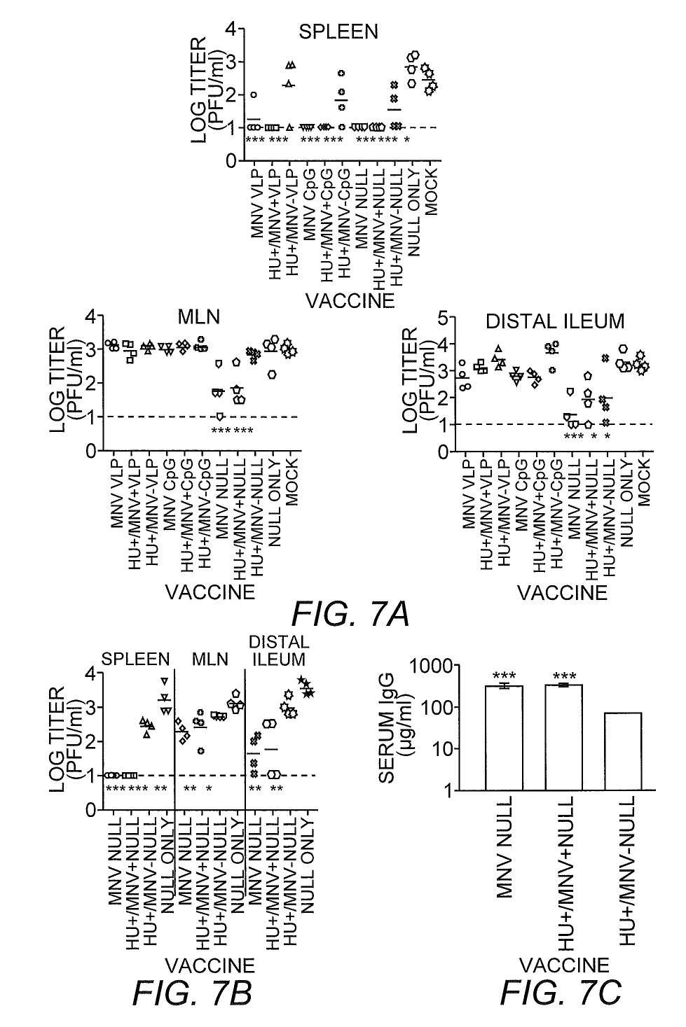

Multivalent VLP vaccines coadministered with null VRPs result in decreased viral load following MNV challenge. To determine if monovalent and multivalent vaccines can protect against norovirus challenge, we utilized the MNV infection model. Mice were immunized with monovalent MNV VLP vaccines or multivalent VLP vaccines consisting of eight human VLPs with MNV VLPs (Hu+/MNV+) or without MNV VLPs (Hu+/MNV−) (Table 1). Each was administered alone or in conjunction with CpG or null VRP adjuvants, same as the way human strain vaccines were administered, as described above. Mice were then challenged with MNV 3 weeks after secondary immunization, and spleens, MLNs, and distal ileums were harvested 3 days later. Viral titers of tissue homogenates were determined by plaque assay. Monovalent and MNV+/Hu+ vaccination with or without adjuvant induced complete protection from MNV infection in the spleen, with significantly lower viral titers than those induced by vaccination with null VRP alone (P<0.001) (FIG. 7A). Hu+/MNV− vaccination did not completely protect against MNV infection in the spleen; however, viral loads were significantly lower in Hu+/MNV− groups coadministered with null VRP adjuvant than in those vaccinated with null VRP alone (P<0.05). In contrast, viral loads in MLNs and distal ileum were not significantly reduced following monovalent or multivalent VLP or CpG vaccination compared to those with unvaccinated controls. Null VRP administration, however, significantly reduced viral loads compared to controls following monovalent and Hu+/MNV+ vaccination in both MLNs (P<0.001) and distal ileum (P<0.05). Hu+/MNV− vaccination coadministered with null VRP significantly reduced viral loads in the distal ileum as well (P<0.05). Vaccination and MNV challenge experiments were repeated in the null VRP adjuvant groups only (FIG. 7B) and resulted in similarly reduced viral loads in the MNV and Hu+/MNV+ vaccine groups in the spleen (P<0.001), MLNs (P<0.05), and distal ileum (P<0.01) and reduced loads in the spleen of the Hu+/MNV− vaccine group (P<0.01). MNV, Hu+/MNV+, and Hu+/MNV− antisera all contained MNV-reactive IgG antibodies following null VRP vaccination, where MNV and Hu+/MNV+ responses were equivalent and significantly higher than the cross-reactive response in Hu+/MNV− groups (P<0.001) (FIG. 7C). These findings show that multivalent VRP vaccines can successfully limit the spread of norovirus infection to some peripheral tissues and can reduce viral loads in primary and additional secondary sites of replication even without the presence of homologous MNV antigen in the vaccine composition using the MNV infection model. These results lend strong support for the development of multivalent human norovirus vaccines.

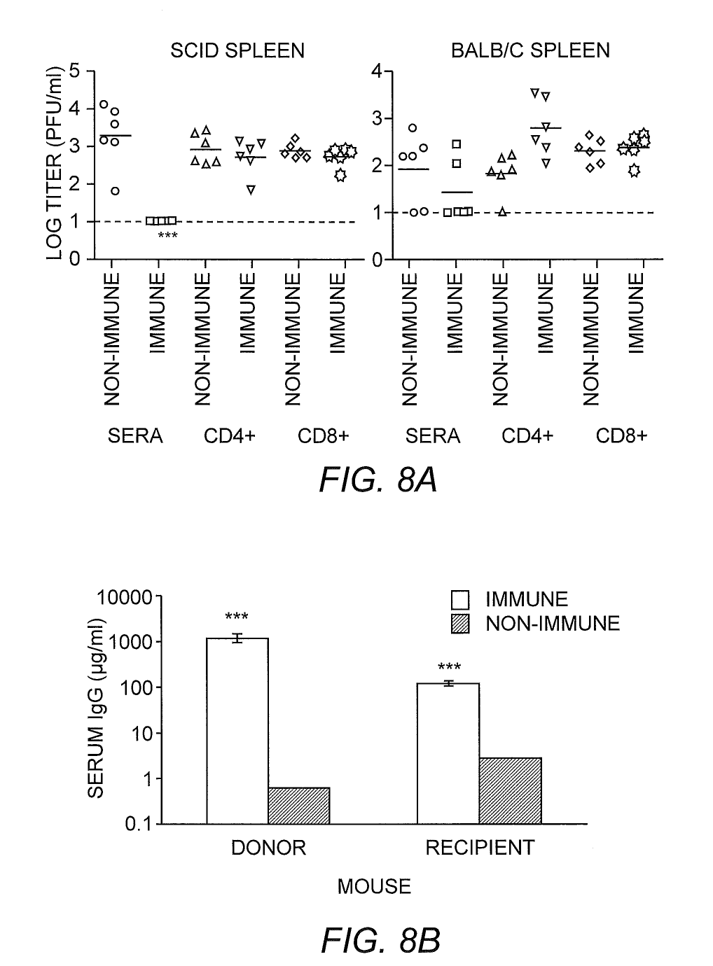

Humoral immunity protects against acute MNV infection. To determine the mechanism of protection induced by null VRP vaccines, we vaccinated immunocompetent wild-type mice monovalently with MNV VLPs coadministered with null VRPs and passively transferred antisera or adoptively transferred purified CD4⁺ or CD8⁺ splenocytes into naïve wild-type mice or immunodeficient SCID mice. Unimmunized mice were treated in parallel as controls. CD4⁺/CD3⁺ and CD8⁺/CD3⁺ T cells from immune and nonimmune spleens were each found to be ≧90% pure by FACS analysis (data not shown). After 24 h, transfer recipient mice were infected with MNV, and tissues were harvested 3 days later. Adoptive transfers of immune or nonimmune CD4⁺ or CD8⁺ splenocytes did not prevent establishment of MNV infection in the spleens of wild-type or SCID mice, as determined by plaque assay (FIG. 8A). Passive transfer of antisera, however, was able to protect SCID mice from MNV infection in the spleen in all mice tested, whereas transfer of nonimmune sera had no effect on viral titers (P<0.001) (FIG. 8A). Wild-type mice also exhibited reduced viral loads in the spleen following passive transfer of immune sera compared to those exhibited following passive transfer of nonimmune sera, with a number of animals cleared of detectable virus. Significant MNV-specific antibodies were found to be circulating in both donor wild-type mice and recipient mice but not in nonimmune controls (P<0.001) (FIG. 8B). Viral titers in the MLNs and distal ileum of wild-type recipient mice were not reduced following transfer of immune sera, CD4⁺ T cells, or CD8⁺ T cells compared to those following nonimmune transfers (data not shown). Because SCID mice do not maintain a competent adaptive immune system and have underdeveloped immune organs, MLNs were not analyzed in this group. Furthermore, SCID mice did not support measurable viral titers in the distal ileum in either transfer group (data not shown). Together, however, these data clearly indicate that humoral immunity induced by monovalent null VRP vaccination can prevent establishment of acute MNV infection and provide further support for the development of null VRP vaccines in humans.

### EXAMPLES

### Summary

We have systematically designed and tested the efficacy of monovalent and multivalent norovirus VLP vaccines coadministered with null VRP adjuvants in generating cross-reactive and receptor-blocking antibody responses and protection against heterologous MNV challenge. These findings are supported by evidence showing that (i) immunodeficient mice were completely protected against MNV infection following transfer of antisera from wild-type mice following monovalent MNV VLP vaccination coadministered with null VRP adjuvant, most likely by antibody-mediated neutralization; (ii) increasing the number of antigens in the vaccine composition did not significantly blunt the immune response to the original antigens; (iii) VLP vaccines lacking target antigens induced strong cross-reactive antibody responses to heterologous strains that partially blocked receptor binding to these strains; and (iv) VRP-adjuvanted VLP vaccines lacking target antigens significantly reduced viral loads in murine tissues following heterologous viral challenge. Although multivalent vaccination did not provide protection from heterologous MNV infection, the significant reduction in observed viral load may be tightly correlated with reduction of clinical disease, as seen with human immunodeficiency virus (HIV), respiratory syncytial virus, or human papillomavirus infections (7, 14, 55), or may alter transmission rates following infection. In general, our data support the development of multivalent VLP/null VRP vaccines against highly heterogeneous noroviruses.

Overall, these studies indicate that increased antibody cross-reactivity to heterologous norovirus strains following multivalent VLP vaccination coadministered with null VRP adjuvant may significantly protect against subsequent norovirus infection. Homologous vaccination induced antibodies that completely blocked receptor binding and was able to completely protect against infection in transfer experiments. Multivalent vaccines also induced robust cross-reactive antibody blockade responses, concentrated mucosal IgG, and limited viral loads following MNV challenge. Unfortunately, mice do not develop clinical disease after MNV challenge making it impossible to determine if reduced viral loads in vaccinated mice correspond to reduced morbidity. Similar experiments with swine can address this issue directly. However, the efficacy of norovirus vaccine formulations containing multiple distinct VLP antigens is supported by our findings that incorporation of up to nine different norovirus strains did not detract from the overall specific immune response generated to each individual antigen; thus, more significant protection might be afforded against the vaccine strains included in the cocktail. Currently, human VLP vaccines containing GII.4 components are widely needed to prevent frequent norovirus outbreaks; however, multivalent vaccines containing additional GI and GII components may be advantageous in preventing further isolated outbreaks and emergence of new predominant strains. The data presented in this study support our conclusion that multivalent norovirus VLP vaccines supplemented with VRP adjuvant will likely provide a safe and effective platform for controlling norovirus infections in humans.

TABLE 1
VLP vaccination chart

| VLP | | | VLP(s) in vaccine |
|---|---|---|---|
| vaccine | Genogroup(s) | Type | composition^{a} |
| NV | GI | Monovalent | NV |
| GI+ | | Multivalent | NV, SoV, DSV, Chiba |
| GI− | | Multivalent | SoV, DSV, Chiba (−) NV |
| LV | GII | Monovalent | LV |
| GII+ | | Multivalent | LV, HV, TV, M7 |
| GII− | | Multivalent | HV, TV, M7 (−) LV |
| GI+/GII+ | GI/GII | Multivalent | All GI/GII |
| GI−/GII− | | Multivalent | All GI/GII (−) NV/LV |
| MNV | GV | Monovalent | MNV |
| Hu+/MNV+ | GI/GII/GV | Multivalent | All GI/GII, MNV |
| Hu+/MNV− | GI/GII | Multivalent | All GI/GII (−) MNV |

^{a}VLP-associated strains and years of isolation: NV, 1968; SoV, 1999; DSV, 1999; Chiba, 2000; LV, 1997; Hawaii virus (HV), 1971; TV, 1999; M7,1999; MNV, 2003.
− in parentheses indicates vaccines formulated without the following listed VLPs.

TABLE 2
Average percent sera for blockade of 50%
(BT50) and 90% (BT90) H type 3 binding

| | Avg % sera (range) for blockade of H type 3 binding to^{a}: | | | | | | | |
|---|---|---|---|---|---|---|---|---|
| | NV VLP | | | | LV VLP | | | |
| Vaccine | BT50 | | BT90 | | BT50 | | BT90 | |
| NV VLP | 2.2 | (0.6-5) | 6.9 | (1.3-20) | 20 | | 20 | |
| NV CpG | 0.5 | (0.2-0.6) | 1.4 | (0.6-2.5) | 20 | | 20 | |
| NV null | 0.2 | (0.2-0.6) | 0.4 | (0.2-1.3) | 20 | | 20 | |
| LV VLP | 20 | | 20 | | 6.3 | (2.5-10) | 12.5 | (5-20) |
| LV CpG | 20 | | 20 | | 1.0 | (0.2-2.5) | 2.0 | (0.6-5) |
| LV null | 20 | | 20 | | 0.2 | | 0.4 | (0.2-1.3) |
| GI+ null | 0.8 | (0.6-1.3) | 1.7 | (1.3-2.5) | 20 | | 20 | |
| GI− null | 12.5 | (10-20) | 20 | | 20 | | 20 | |
| GII+ null | 20 | | 20 | | 0.2 | | 0.3 | (0.2-0.6) |
| GII− null | 20 | | 20 | | 20 | | 20 | |
| GI+/GII+ VLP | 2.9 | (1.3-5) | 7.7 | (1.3-10) | 1.5 | (0.2-5) | 17.5 | (10-20) |

| | | | | | | | |
|---|---|---|---|---|---|---|---|
| GI+/GII+ CpG | 0.6 | (0.2-1.3) | 1.8 | (0.6-2.5) | 0.2 | 0.3 | (0.2-0.6) |

| GI+/GII+ null | 0.8 | (0.6-1.3) | 1.7 | (1.3-2.5) | 0.2 | | 0.2 |
|---|---|---|---|---|---|---|---|
| GI−/GII− VLP | 20 | | 20 | | 20 | | 20 |
| GI−/GII− CpG | 8.0 | (0.2-20) | 18 | (10-20) | 17.5 | (10-20) | 20 |

| | | | | | | |
|---|---|---|---|---|---|---|
| GI−/GII− null | 7.1 | (2.5-10) | 20 | 8.8 | (2.5-20) | 20 |

^{a}Sera that blocked H type 3 binding at the lowest concentration tested were assigned a BT value that is half the lowest serum concentration tested (0.2%). Sera that could not block H type 3 binding at the highest concentration tested were assigned a BT value that is twice the highest serum concentration tested (20%).

TABLE 3
Anti-NV IgG and IgA in fecal extracts^{a}

| | Anti-NV | Total | Anti- | Anti-NV | Total | Anti- |
|---|---|---|---|---|---|---|
| | IgG ± SEM | IgG ± SEM | NV/total | IgA ± SEM | IgA ± SEM | NV/total |
| Vaccine^{b} | (ng/ml) | (ng/ml)^{d} | IgG (%)^{e} | (ng/ml) | (mg/ml)^{d} | IgA (%)^{e} |
| VLP | | | | | | |
| NV | 0.5 ± 0.3^{c} | 62.3 ± 12.6 | 0.8 | 2.5 ± 1.2 | 47.3 ± 10 | 5.3E−03 |
| GI/GII+ | 2.4 ± 0.7 | 174.6 ± 79.1 | 1.4 | 0.7 ± 0.1 | 25.7 ± 4.6 | 2.7E−03 |
| GI/GII− | 0.5 ± 0.2 | 110.7 ± 30.6 | 0.5 | 1.1 ± 0.7 | 30.6 ± 2.6 | 3.6E−03 |
| CpG | | | | | | |
| NV | 12.2 ± 6.7 | 94.5 ± 9.7 | 12.9 | 7.2 ± 2.2 | 56.5 ± 2.0 | 1.3E−02 |
| GI/GII+ | 34.9 ± 8.0 | 316.0 ± 203.8 | 11.0 | 3.2 ± 1.9 | 30.6 ± 8.1 | 1.0E−02 |
| GI/GII− | 10.4 ± 6.6 | 315.8 ± 6.0 | 3.3 | 1.8 ± 0.8 | 31.3 ± 1.5 | 5.8E−03 |
| Null VRP | | | | | | |
| NV | 44.1 ± 9.3 | 254.0 ± 54.9 | 17.4 | 68.1 ± 32.2 | 44.0 ± 4.7 | 1.5E−01 |
| GI/GII+ | 88.1 ± 47.9 | 504.6 ± 267.4 | 17.5 | 10.4 ± 8.7 | 27.5 ± 1.6 | 3.8E−02 |
| GI/GII− | 9.1 ± 4.2 | 318.3 ± 50.6 | 2.9 | 7.7 ± 2.4 | 31.5 ± 7.6 | 2.4E−02 |

^{a}See text for statistical analysis.
^{b}Adjuvants (if applicable) coadministered with VLP vaccines are listed, and VLP vaccine groups are listed beneath each adjuvant.
^{c}Lower limit of detection for IgG and IgA assays is 0.2 ng/ml.
^{d}Total Ig concentration in sample; nonspecific for antigen.
^{e}The percentage of NV-specific antibody per total antibody was calculated as the anti-NV Ig concentration/total nonspecific Ig concentration × 100.

### REFERENCES

1. Aggarwal, P., R. M. Pandey, and P. Seth. 2005. Augmentation of HIV-1 subtype C vaccine constructs induced immune response in mice by CpG motif 1826-ODN. Viral Immunol. 18:213-223.
2. Baker, C. J., M. A. Rench, M. Fernandez, L. C. Paoletti, D. L. Kasper, and M. S. Edwards. 2003. Safety and immunogenicity of a bivalent group B streptococcal conjugate vaccine for serotypes II and III. J. Infect. Dis. 188:66-73.
3. Ball, J. M., M. K. Estes, M. E. Hardy, M. E. Conner, A. R. Opekun, and D. Y. Graham. 1996. Recombinant Norwalk virus-like particles as an oral vaccine, Arch. Virol. Suppl. 12:243-249.
4. Ball, J. M., D. Y. Graham, A. R. Opekun, M. A. Gilger, R. A. Guerrero, and M. K. Estes. 1999. Recombinant Norwalk virus-like particles given orally to volunteers: phase I study. Gastroenterology 117:40-48.
5. Ball, J. M., M. E. Hardy, R. L. Atmar, M. E. Conner, and M. K. Estes. 1998. Oral immunization with recombinant Norwalk virus-like particles induces a systemic and mucosal immune response in mice. J. Virol. 72:1345-1353.
6. Baric, R. S., B. Yount, L. Lindesmith, P. R. Harrington, S. R. Greene, F. C. Tseng, N. Davis, R. E. Johnston, D. G. Klapper, and C. L. Moe. 2002. Expression and self-assembly of Norwalk virus capsid protein from Venezuelan equine encephalitis virus replicons. J. Virol. 76:3023-3030.
7. Broccolo, F., and C. E. Cocuzza. 2008. Automated extraction and quantitation of oncogenic HPV genotypes from cervical samples by a real-time PCR-based system. J. Virol. Methods 148:48-57.
8. Chachu, K. A., D. W. Strong, A. D. LoBue, C. E. Wobus, R. S. Baric, and H. W. Virgin IV. 2008. Antibody is critical for the clearance of murine norovirus infection. J. Virol. 82:6610-6617.
9. Cheetham, S., M. Souza, T. Meulia, S. Grimes, M. G. Han, and L. J. Saif. 2006. Pathogenesis of a genogroup II human norovirus in gnotobiotic pigs. J. Virol. 80:10372-10381.
10. Cho, M. W., Y. B. Kim, M. K. Lee, K. C. Gupta, W. Ross, R. Plishka, A. Buckler-White, T. Igarashi, T. Theodore, R. Byrum, C. Kemp, D. C. Montefiori, and M. A. Martin. 2001. Polyvalent envelope glycoprotein vaccine elicits a broader neutralizing antibody response but is unable to provide sterilizing protection against heterologous simian/human immunodeficiency virus infection in pigtailed macaques. J. Virol. 75:2224-2234.
11. Chu, R. S., O. S. Targoni, A. M. Krieg, P. V. Lehmann, and C. V. Harding. 1997. CpG oligodeoxynucleotides act as adjuvants that switch on T helper 1 (Th1) immunity. J. Exp. Med. 186:1623-1631.
12. Davis, N. L., A. West, E. Reap, G. MacDonald, M. Collier, S. Dryga, M. Maughan, M. Connell, C. Walker, K. McGrath, C. Cecil, L. H. Ping, J. Frelinger, R. Olmsted, P. Keith, R. Swanstrom, C. Williamson, P. Johnson, D. Montefiori, and R. E. Johnston. 2002. Alphavirus replicon particles as candidate HIV vaccines. IUBMB Life 53:209-211.
13. Garland, S. M., M. Steben, M. Hernandez-Avila, L. A. Koutsky, C. M. Wheeler, G. Perez, D. M. Harper, S. Leodolter, G. W. Tang, D. G. Ferris, M. T. Esser, S. C. Vuocolo, M. Nelson, R. Railkar, C. Sattler, and E. Barr. 2007. Noninferiority of antibody response to human papillomavirus type 16 in subjects vaccinated with monovalent and quadrivalent L1 virus-like particle vaccines. Clin. Vaccine Immunol. 14:792-795.
14. Gerna, G., G. Campanini, V. Rognoni, A. Marchi, F. Rovida, A. Piralla, and E. Percivalle. 2008. Correlation of viral load as determined by real-time RT-PCR and clinical characteristics of respiratory syncytial virus lower respiratory tract infections in early infancy. J. Clin. Virol. 41:45-48.
15. Green, K. Y., R. M. Chanock, and A. Z. Kapikian. 2001. Human caliciviruses, p. 841-874. In D. M. Knipe and P. M. Howley (ed.), Fields virology, fourth ed., vol. 1. Lippincott Williams & Wilkins, Philadelphia, Pa.
16. Guerrero, R. A., J. M. Ball, S. S. Krater, S. E. Pacheco, J. D. Clements, and M. K. Estes. 2001. Recombinant Norwalk virus-like particles administered intranasally to mice induce systemic and mucosal (fecal and vaginal) immune responses. J. Virol. 75:9713-9722.
17. Hale, A. D., D. C. Lewis, X. Jiang, and D. W. Brown. 1998. Homotypic and heterotypic IgG and IgM antibody responses in adults infected with small round structured viruses. J. Med. Virol. 54:305-312.
18. Harrington, P. R., L. Lindesmith, B. Yount, C. L. Moe, and R. S. Baric. 2002. Binding of Norwalk virus-like particles to ABH histo-blood group antigens is blocked by antisera from infected human volunteers or experimentally vaccinated mice. J. Virol. 76:12335-12343.
19. Harrington, P. R., B. Yount, R. E. Johnston, N. Davis, C. Moe, and R. S. Baric. 2002. Systemic, mucosal, and heterotypic immune induction in mice inoculated with Venezuelan equine encephalitis replicons expressing Norwalk virus-like particles. J. Virol. 76:730-742.
20. Hu, M. C., M. A. Walls, S. D. Stroop, M. A. Reddish, B. Beall, and J. B. Dale. 2002. Immunogenicity of a 26-valent group A streptococcal vaccine. Infect. Immun. 70:2171-2177.
21. Huang, Z., G. Elkin, B. J. Maloney, N. Beuhner, C. J. Arntzen, Y. Thanavala, and H. S. Mason. 2005. Virus-like particle expression and assembly in plants: hepatitis B and Norwalk viruses. Vaccine 23:1851-1858.
22. Humphries, H. E., J. N. Williams, R. Blackstone, K. A. Jolley, H. M. Yuen, M. Christodoulides, and J. E. Heckels. 2006. Multivalent liposome-based vaccines containing different serosubtypes of PorA protein induce cross-protective bactericidal immune responses against Neisseria meningitidis. Vaccine 24:36-44.
23. Johnson, P. C., J. J. Mathewson, H. L. DuPont, and H. B. Greenberg. 1990. Multiple-challenge study of host susceptibility to. Norwalk gastroenteritis in U.S. adults. J. Infect. Dis. 161:18-21.
24. Karst, S. M., C. E. Wobus, M. Lay, J. Davidson, and H. W. Virgin. 2003. STAT1-dependent innate immunity to a Norwalk-like virus. Science 299:1575-1578.
25. Klinman, D. M., D. Currie, I. Gursel, and D. Verthelyi. 2004. Use of CpG oligodeoxynucleotides as immune adjuvants. Immunol. Rev. 199:201-216.
26. Kotloff, K. L., M. Corretti, K. Palmer, J. D. Campbell, M. A. Reddish, M. C. Hu, S. S. Wasserman, and J. B. Dale. 2004. Safety and immunogenicity of a recombinant multivalent group a streptococcal vaccine in healthy adults: phase 1 trial. JAMA 292:709-715.
27. Krieg, A. M., A. K. Yi, and G. Hartmann. 1999. Mechanisms and therapeutic applications of immune stimulatory cpG DNA. Pharmacol. Ther. 84:113-120.
28. Lindesmith, L., C. Moe, J. Lependu, J. A. Frelinger, J. Treanor, and R. S. Baric. 2005. Cellular and humoral immunity following Snow Mountain virus challenge. J. Virol. 79:2900-2909.
29. Lindesmith, L., C. Moe, S. Marionneau, N. Ruvoen, X. Jiang, L. Lindblad, P. Stewart, J. LePendu, and R. Baric. 2003. Human susceptibility and resistance to Norwalk virus infection. Nat. Med. 9:548-553.
30. Lindesmith, L. C., E. F. Donaldson, A. D. Lobue, J. L. Cannon, D. P. Zheng, J. Vinje, and R. S. Baric. 2008. Mechanisms of GII.4 norovirus persistence in human populations. PLoS Med. 5:e31.
31. LoBue, A. D., L. Lindesmith, B. Yount, P. R. Harrington, J. M. Thompson, R. E. Johnston, C. L. Moe, and R. S. Baric. 2006. Multivalent norovirus vaccines induce strong mucosal and systemic blocking antibodies against multiple strains. Vaccine 24:5220-5234.
32. Marionneau, S., N. Ruvoen, B. Le Moullac-Vaidye, M. Clement, A. Cailleau-Thomas, G. Ruiz-Palacois, P. Huang, X. Jiang, and J. Le Pendu. 2002. Norwalk virus binds to histo-blood group antigens present on gastroduodenal epithelial cells of secretor individuals. Gastroenterology 122:1967-1977.
33. Mead, P. S., L. Slutsker, V. Dietz, L. F. McCaig, J. S. Bresee, C. Shapiro, P. M. Griffin, and R. V. Tauxe. 1999. Food-related illness and death in the United States. Emerg. Infect. Dis. 5:607-625.
34. Nicollier-Jamot, B., A. Ogier, L. Piroth, P. Pothier, and E. Kohli. 2004. Recombinant virus-like particles of a norovirus (genogroup II strain) administered intranasally and orally with mucosal adjuvants LT and LT(R192G) in BALB/c mice induce specific humoral and cellular Th1/Th2-like immune responses. Vaccine 22:1079-1086.
35. Noel, J. S., T. Ando, J. P. Leite, K. Y. Green, K. E. Dingle, M. K. Estes, Y. Seto, S. S. Monroe, and R. I. Glass. 1997. Correlation of patient immune responses with genetically characterized small round-structured viruses involved in outbreaks of nonbacterial acute gastroenteritis in the United States, 1990 to 1995. J. Med. Virol. 53:372-383.
36. Ozawa, K., T. Oka, N. Takeda, and G. S. Hansman. 2007. Norovirus infections in symptomatic and asymptomatic food handlers in Japan. J. Clin. Microbiol. 45:3996-4005.
37. Parrino, T. A., D. S. Schreiber, J. S. Trier, A. Z. Kapikian, and N. R. Blacklow. 1977. Clinical immunity in acute gastroenteritis caused by Norwalk agent. N. Engl. J. Med. 297:86-89.
38. Patel, M. M., M. A. Widdowson, R. I. Glass, K. Akazawa, J. Vinje, and U. D. Parashar. 2008. Systematic literature review of role of noroviruses in sporadic gastroenteritis. Emerg. Infect. Dis. 14:1224-1231.
39. Periwal, S. B., K. R. Kourie, N. Ramachandaran, S. J. Blakeney, S. DeBruin, D. Zhu, T. J. Zamb, L. Smith, S. Udem, J. H. Eldridge, K. E. Shroff, and P. A. Reilly. 2003. A modified cholera holotoxin CT-E29H enhances systemic and mucosal immune responses to recombinant Norwalk virus-virus like particle vaccine. Vaccine 21:376-385.
40. Platt, R., C. Coutu, T. Meinert, and J. A. Roth. 2008. Humoral and T cell-mediated immune responses to bivalent killed bovine viral diarrhea virus vaccine in beef cattle. Vet. Immunol. Immunopathol. 122:8-15.
41. Pushko, P., M. Bray, G. V. Ludwig, M. Parker, A. Schmaljohn, A. Sanchez, P. B. Jahrling, and J. F. Smith. 2000. Recombinant RNA replicons derived from attenuated Venezuelan equine encephalitis virus protect guinea pigs and mice from Ebola hemorrhagic fever virus. Vaccine 19:142-153.
42. Pushko, P., M. Parker, G. V. Ludwig, N. L. Davis, R. E. Johnston, and J. F. Smith. 1997. Replicon-helper systems from attenuated Venezuelan equine encephalitis virus: expression of heterologous genes in vitro and immunization against heterologous pathogens in vivo. Virology 239:389-401.
43. Rockx, B., R. S. Baric, I. de Grijs, E. Duizer, and M. P. Koopmans. 2005. Characterization of the homo- and heterotypic immune responses after natural norovirus infection. J. Med. Virol. 77:439-446.
44. Roda, J. M., R. Parihar, and W. E. Carson III. 2005. CpG-containing oligodeoxynucleotides act through TLR9 to enhance the NK cell cytokine response to antibody-coated tumor cells. J. Immunol. 175:1619-1627.
45. Sharma, R., and C. L. Sharma. 2007. Quadrivalent human papillomavirus recombinant vaccine: the first vaccine for cervical cancers. J. Cancer Res. Ther. 3:92-95.
46. Souza, M., V. Costantini, M. S. Azevedo, and L. J. Saif. 2007. A human norovirus-like particle vaccine adjuvanted with ISCOM or mLT induces cytokine and antibody responses and protection to the homologous GII.4 human norovirus in a gnotobiotic pig disease model. Vaccine 25:8448-8459.
47. Tacket, C. O., H. S. Mason, G. Losonsky, M. K. Estes, M. M. Levine, and C. J. Arntzen. 2000. Human immune responses to a novel Norwalk virus vaccine delivered in transgenic potatoes. J. Infect. Dis. 182:302-305.
48. Tacket, C. O., M. B. Sztein, G. A. Losonsky, S. S. Wasserman, and M. K. Estes. 2003. Humoral, mucosal, and cellular immune responses to oral Norwalk virus-like particles in volunteers. Clin. Immunol. 108:241-247.
49. Thompson, J. M., M. G. Nicholson, A. C. Whitmore, M. Zamora, A. West, A. Iwasaki, H. F. Staats, and R. E. Johnston. 2008. Nonmucosal alphavirus vaccination stimulates a mucosal inductive environment in the peripheral draining lymph node. J. Immunol. 181:574-585.
50. Thompson, J. M., A. C. Whitmore, J. L. Konopka, M. L. Collier, E. M. Richmond, N. L. Davis, H. F. Staats, and R. E. Johnston. 2006. Mucosal and systemic adjuvant activity of alphavirus replicon particles. Proc. Natl. Acad. Sci. USA 103:3722-3727.
51. Thompson, J. M., A. C. Whitmore, H. F. Staats, and R. E. Johnston. 2008. Alphavirus replicon particles acting as adjuvants promote CD8⁺ T cell responses to co-delivered antigen. Vaccine 26:4267-4275.
52. Thornburg, N. J., C. A. Ray, M. L. Collier, H. X. Liao, D. J. Pickup, and R. E. Johnston. 2007. Vaccination with Venezuelan equine encephalitis replicons encoding cowpox virus structural proteins protects mice from intranasal cowpox virus challenge. Virology 362:441-452.
53. Treanor, J. J., X. Jiang, H. P. Madore, and M. K. Estes. 1993. Subclass-specific serum antibody responses to recombinant Norwalk virus capsid antigen (rNV) in adults infected with Norwalk, Snow Mountain, or Hawaii virus. J. Clin. Microbiol. 31:1630-1634.
54. Trollfors, B., J. Taranger, T. Lagergard, and V. Sundh. 2005. Reduced immunogenicity of diphtheria and tetanus toxoids when combined with pertussis toxoid. Pediatr. Infect. Dis. J. 24:85-86.
55. UK Collaborative HIV Cohort (CHIC) Study Steering Committee. 2007. HIV diagnosis at CD4 count above 500 cells/mm³ and progression to below 350 cells/mm³ without antiretroviral therapy. J. Acquir. Immune Defic. Syndr. 46:275-278.
56. Wobus, C. E., S. M. Karst, L. B. Thackray, K. O. Chang, S. V. Sosnovtsev, G. Belliot, A. Krug, J. M. Mackenzie, K. Y. Green, and H. W. Virgin. 2004. Replication of norovirus in cell culture reveals a tropism for dendritic cells and macrophages. PLoS Biol. 2:e432.
57. Wyatt, R. G., R. Dolin, N. R. Blacklow, H. L. DuPont, R. F. Buscho, T. S. Thornhill, A. Z. Kapikian, and R. M. Chanock. 1974. Comparison of three agents of acute infectious nonbacterial gastroenteritis by cross-challenge in volunteers. J. Infect. Dis. 129:709-714.
58. Xia, M., T. Farkas, and X. Jiang. 2007. Norovirus capsid protein expressed in yeast forms virus-like particles and stimulates systemic and mucosal immunity in mice following an oral administration of raw yeast extracts. J. Med. Virol. 79:74-83.
59. Zhang, X., N. A. Buehner, A. M. Hutson, M. K. Estes, and H. S. Mason. 2006. Tomato is a highly effective vehicle for expression and oral immunization with Norwalk virus capsid protein. Plant Biotechnol. J. 4:419-432.

The foregoing is illustrative of the present invention, and is not to be construed as limiting thereof. The invention is defined by the following claims, with equivalents of the claims to be included therein.

That which is claimed is:

1. An immunogenic formulation comprising virus-like particles (VLPs) from two or more genoclusters and/or strains of norovirus in a pharmaceutically acceptable carrier.

2. The immunogenic formulation of claim 1, wherein the immunogenic formulation induces humoral, mucosal and/or cellular immunity against one or more norovirus genoclusters and/or strains included in the immunogenic formulation.

3. The immunogenic formulation of claim 1, wherein the immunogenic formulation induces humoral, mucosal and/or cellular immunity against one or more norovirus genoclusters and/or strains not included within the immunogenic formulation.

4. The immunogenic formulation of claim 1, wherein the immunogenic formulation includes a VLP from a GI norovirus genocluster.

5. The immunogenic formulation of claim 4, wherein the immunogenic formulation induces humoral, mucosal and/or cellular immunity against one or more GI norovirus genoclusters and/or strains not included within the immunogenic formulation.

6. The immunogenic formulation of claim 1, wherein the immunogenic formulation includes a VLP from a GII norovirus genocluster.

7. The immunogenic formulation of claim 6, wherein the immunogenic formulation induces humoral, mucosal and/or cellular immunity against one or more GII norovirus genoclusters and/or strains not included within the immunogenic formulation.

8. The immunogenic formulation of claim 1, wherein the immunogenic formulation includes a VLP from a GI norovirus genocluster and a VLP from a GII norovirus genocluster.

9. The immunogenic formulation of claim 8, wherein the immunogenic formulation induces humoral, mucosal and/or cellular immunity against one or more GI norovirus genoclusters and/or strains not included within the immunogenic formulation and one or more GII norovirus genoclusters and/or strains not included within the formulation.

10. The immunogenic formulation of claim 1, wherein the immunogenic formulation further comprises an adjuvant.

11. The immunogenic formulation of claim 10, wherein the adjuvant comprises CpG.

12. The immunogenic formulation of claim 10, wherein the adjuvant comprises an alphavirus adjuvant comprising:
a modified alphavirus genomic nucleic acid that lacks sequences encoding the alphavirus structural proteins required for production of new alphavirus particles;
wherein the modified alphavirus genome does not comprise a heterologous nucleic acid sequence encoding the VLP from two or more genoclusters and/or strains of norovirus.

13. The immunogenic formulation of claim 12, wherein the modified alphavirus genome does not comprise a heterologous nucleic acid sequence encoding a polypeptide of interest or a functional untranslated RNA.

14. The immunogenic formulation of claim 12, wherein the alphavirus adjuvant is replication-competent.

15. The immunogenic formulation of claim 12, wherein the alphavirus adjuvant comprises a propagation-defective alphavirus particle that further comprises an alphavirus virion coat that packages the modified alphavirus genomic nucleic acid.

16. The immunogenic formulation of claim 12, wherein the modified alphavirus genomic nucleic acid does not comprise a heterologous nucleic acid sequence.

17. The immunogenic formulation of claim 12, wherein the 26S promoter is deleted from the modified alphavirus genomic nucleic acid or is a 26S promoter having reduced transcriptional activity.

18. The immunogenic formulation of claim 12, wherein the alphavirus adjuvant is attenuated.

19. The immunogenic formulation of claim 12, wherein the modified alphavirus genomic nucleic acid is a modified Venezuelan Equine Encephalitis (VEE) viral genomic nucleic acid.

20. The immunogenic formulation of claim 19, wherein the alphavirus adjuvant comprises a propagation-defective VEE particle that further comprises a VEE virion coat that packages the VEE viral genomic nucleic acid.

21. The immunogenic formulation of claim 20, wherein the 26S promoter is deleted from the modified VEE genomic nucleic acid or is a 26S promoter having reduced transcriptional activity.

22. A method of producing an immune response against two or more noroviruses in a subject, the method comprising administering an immunogenically effective amount of the formulation of claim 1 to the subject.

23. A method of protecting a subject from infection by two or more noroviruses, the method comprising administering the formulation of claim 1 to the subject in an amount effective to protect the subject from infection by the two or more noroviruses.

24. A method of producing an immune response against two or more noroviruses in a subject, the method comprising administering to the subject:
(a) an immunogenically effective amount of the formulation of claim 1; and
(b) an alphavirus adjuvant comprising: a modified alphavirus genomic nucleic acid that lacks sequences encoding the alphavirus structural proteins required for production of new alphavirus particles; wherein the modified alphavirus genome does not comprise a heterologous nucleic acid sequence encoding the VLP from two or more genoclusters and/or strains of norovirus.

25. A method of protecting a subject from infection by two or more noroviruses, the method comprising administering to the subject:
(a) the formulation of claim 1 in an amount effective to protect the subject from infection by the two or more noroviruses; and
(b) an alphavirus adjuvant comprising: a modified alphavirus genomic nucleic acid that lacks sequences encoding the alphavirus structural proteins required for production of new alphavirus particles; wherein the modified alphavirus genome does not comprise a heterologous nucleic acid sequence encoding the VLP from two or more genoclusters and/or strains of norovirus.

26. The method of claim 22, wherein the subject is a human subject.

27. The method of claim 22, wherein the subject is an immunocompromised subject.

28. The method of claim 22, wherein the subject is a geriatric subject.

29. The method of claim 28, wherein the subject is living in an institutional setting.

30. The method of claim 22, wherein the subject is an infant.

31. The method of claim 22, wherein the subject is a child under the age of 5.

32. The method of claim 22, wherein the subject is a member of the military.
